(19) United States
(12) Patent Application Publication
(10) Pub. No.: US 2021/0335462 A1
(43) Pub. Date: Oct. 28, 2021

(54) System and Method for Providing Access to Digital Assets Upon Occurrence of a Predetermined Event

(71) Applicant: Paul Michael Yantus, Issaquah, WA (US)

(72) Inventor: Paul Michael Yantus, Issaquah, WA (US)

(21) Appl. No.: 17/242,742

(22) Filed: Apr. 28, 2021

Related U.S. Application Data
(60) Provisional application No. 63/016,754, filed on Apr. 28, 2020.

(51) Int. Cl. G16H 10/60; G16H 80/00; G06F 21/62; G06Q 50/18

(52) U.S. Cl. CPC G16H 10/60; G06Q 50/18; G06F 21/6245; G16H 80/00

(57) ABSTRACT

A system includes a computer system, data storage media and a user application. The user application is associated with a user device. The processing device is in communication with the user application and includes an account creation module, an event creation module and an event trigger module defined by the computer program. The account creation module allows a user to create a user account using the user interface of the user application. The event creation module allows the user to access a user account and to create an event associated with the user account. The event includes an event trigger and digital asset access information associated with a digital asset. The event trigger module performs the steps of receiving an indication that the event trigger has occurred and responsively providing access to the digital asset to a recipient using the digital asset access information.

### RELATED APPLICATION

This application claims priority to and the benefit of U.S. Provisional Patent Application No. 63/016,754, filed Apr. 28, 2020, which is hereby incorporated by reference in its entirety.

### FIELD OF THE INVENTION

The present invention relates to digital assets, and more particularly, to a system and method for providing access to digital assets upon occurrence of a predetermined event trigger.

### BACKGROUND

Historically, important documents, such as financial statements, pictures, newspapers, legal documents, bank records, and other documents were embodied in physical form. The physical documents represented the historical record of important people, families, governmental entities and institutions. These records are imperfect, physical records are lost, destroyed, subject to degradation with the passage of time.

Presently, much of these same records are stored digitally. For example, banking and other forms of financial transactions may be performed exclusively within electronic systems and may never be embodied in physical form or preserved in that manner. Family histories, in the form of pictures, may also be stored all, or mostly, electronically. For example, pictures are typically taken with some form of digital camera and may not be printed in physical form.

These types of records may be stored either locally, on a computer in the photographer's possession or in an external hard disk connected to the photographer's computer or computer system. Alternatively, these records may be stored in external cloud storage (which is typically operated by a third party).

In addition, social networking services are used by a large number of people to: (1) make and interact with friends and others, (2) obtain, and interact with news services, and (3) provide personal and family news to remote family and friends and (4) store certain types of records, such as pictures.

These types of records may, in some ways, be more robust than physical records and may be more easily indexed, identifiable and take up less space. For example, they can be easily be backed up to one or more locations and may be backed up, digitally, to a more physical media, such as optical disks.

However, digital records and accounts do present other challenges. They may become “lost” under different circumstances, e.g., after passage of time or upon certain circumstances, such as the death of the owner. For example, if a single copy of such records exist on a single computer or attached storage device, after the owner's death, another person would have to know of the existence and location of the records in order to obtain and have access to the computer or storage device. Further, if the records are encrypted for privacy purposes, the person attempting to access the records would have to have encryption key.

In addition, for records stored in third party cloud storage facilities or for records or other information stored on social network accounts, after the owner's death, the heir or other person attempting to retrieve or access such records, may have to know of the existence of the records and/or have access to the account information and passwords in order to access. Further, some social networks or services may either lock and/or destroy/delete such records automatically after the passage of a certain amount of time and/or the death of the owner.

The present invention is aimed at one or more of the problems set forth above.

### SUMMARY OF THE INVENTION

In different embodiments of the present invention, systems and methods for managing digital asserts are provided.

In one aspect of the present invention, a digital asset management system including a computer system, data storage media and a user application. The data storage media is coupled to the computer system and stores a computer program defining an algorithm. The user application is coupled to the computer system and being associated with a user device, the user application has a user interface. The processing device is in communication with the user application and including an account creation module, an event creation module and an event trigger module. The account creation module is defined by the computer program and is configured to allow the user to create a user account using the user interface of the user application. The event creation module is defined by the computer program and is configured to allow the user to access a user account and to create an event associated with the user account. The event includes an event trigger and digital asset access information associated with a digital asset. The event trigger module is defined by the computer program and performs the steps of receiving an indication that the event trigger has occurred and responsively providing access to the digital asset to an identified recipient using the digital asset access information.

In another aspect of the present invention, a method for managing digital assets using a computer system, data storage media and a user application is provided. The computer system has a processing device. The data storage media is coupled to the computer system for storing a computer program defining an algorithm. The user application is coupled to the computer system and being associated with a user device. The user application has a user interface. The processing device is in communication with the user application and includes an account creation module, an event creation module and an event trigger module defined by the computer program. The method comprising the steps of allowing the user, using the account creation module, to create a user account using the user interface of the user application and allowing the user, using the event creation module, to access the user account and to create an event associated with the user account, the event including an event trigger and digital asset access information associated with a digital asset. The method further includes the step of establishing, using the event trigger module, an indication that the event trigger has occurred and responsively providing access to the digital asset to a recipient using the digital asset access information.

### BRIEF DESCRIPTION OF THE DRAWINGS

Non-limiting and non-exhaustive embodiments of the present invention are described with reference to the following figures. Other advantages of the present disclosure will be readily appreciated, as the same becomes better understood by reference to the following detailed description when considered in connection with the accompanying drawings.

FIG. 1 is a diagrammatic illustration of a digital asset management system, according to an embodiment of the present invention.

FIG. 2 is a diagrammatic illustration of a computer system associated with the digital asset management system of FIG. 1.

FIG. 3 is a functional diagram of the operation of the digital asset management system of FIG. 1, according to an embodiment of the present invention.

FIG. 4A is a first flow diagram of a sign-up/logon process associated with the digital asset management system of FIG. 1, according to an embodiment of the present invention.

FIG. 4B is a screen shot of an exemplary account sign-up/login dialog associated with the digital asset management system of FIG. 1, according to an embodiment of the present invention.

FIG. 4C is a screen shot of an exemplary account set up dialog associated with the digital asset management system of FIG. 1, according to an embodiment of the present invention.

FIG. 5A is a first portion of a flow diagram associated with the digital asset management system of FIG. 1, according to an embodiment of the present invention.

FIG. 5B is a second portion of the flow diagram of FIG. 5A.

FIG. 5C is a third portion of the flow diagram of FIG. 5A.

FIG. 5D is a functional diagram of the creation of an event in the digital asset management system of FIG. 1, according to an embodiment of the present invention.

FIG. 5E is a screen shot of a first event creation dialog, according to an embodiment of the present invention.

FIG. 5F is a screen shot of a second event creation dialog, according to an embodiment of the present invention.

FIG. 5G is a screen shot of a third event creation dialog, according to an embodiment of the present invention.

FIG. 5H is a screen shot of a fourth event creation dialog, according to an embodiment of the present invention.

FIG. 5I is a screen shot of a fifth event creation dialog, according to an embodiment of the present invention.

FIG. 5J is a screen shot of a sixth event creation dialog, according to an embodiment of the present invention.

FIG. 5K is a screen shot of an exemplary home screen associated with the digital asset management system, according to an embodiment of the present invention.

FIG. 6 is a flow diagram of a second process associated with the digital asset management system, according to an embodiment of the present invention.

FIG. 7 is a flow diagram of a third associated with the digital asset management system, according to an embodiment of the invention.

FIG. 8A is a first portion of a flow diagram associated with the digital asset management system, according to another embodiment of the present invention.

FIG. 8B is a second portion of the flow diagram of FIG. 8A.

FIG. 8C is a flow diagram of a method associated with the digital asset management system of the present invention, according to an embodiment of the present invention.

FIG. 8D is a flow diagram of a method associated with the digital asset management system of the present invention, according to an embodiment of the present invention.

FIG. 8E is a flow diagram of a method associated with the digital asset management system of the present invention, according to an embodiment of the present invention.

FIG. 9A is a representation of an exemplary user data table, according to an embodiment of the present invention.

FIG. 9B is a representation of an exemplary digital asset data table, according to an embodiment of the present invention.

FIG. 10A is a first screen shot of a public website of the digital asset management system, according to an embodiment of the present invention.

FIG. 10B is a second screen shot of the public website of the digital asset management system of FIG. 10A

FIG. 10C is a third screen shot of the public website of the digital asset management system of FIG. 10A.

FIG. 10D is a fourth screen shot of the public website of the digital asset management system of FIG. 10A.

FIG. 10E is a fifth screen shot of the public website of the digital asset management system of FIG. 10A.

FIG. 10F is a sixth screen shot of the public website of the digital asset management system of FIG. 10A.

FIG. 10G is a seventh screen shot of the public website of the digital asset management system of FIG. 10A.

FIG. 10H is an eighth screen shot of the public website of the digital asset management system of FIG. 10A.

FIG. 10I is a ninth screen shot of the public website of the digital asset management system of FIG. 10A.

FIG. 10J is a tenth screen shot of the public website of the digital asset management system of FIG. 10A.

FIG. 10K is an eleventh screen shot of the public website of the digital asset management system of FIG. 10A.

FIG. 10L is a twelfth screen shot of the public website of the digital asset management system of FIG. 10A.

FIG. 11A is a first screen shot of a home page of a digital asset management system of FIG. 10A.

FIG. 11B is a second screen shot of the home page of a digital asset management system of FIG. 11A.

FIG. 11C is a third screen shot of the home page of a digital asset management system of FIG. 11A.

FIG. 11D is a fourth screen shot of the home page of a digital asset management system of FIG. 11A.

FIG. 12A is a first screen shot of an end of life plan worksheet according to an embodiment of the present invention.

FIG. 12B is a second screen shot of the end of life plan worksheet of FIG. 12A.

FIG. 12C is a third screen shot of the end of life plan worksheet of FIG. 12A.

FIG. 12D is a fourth screen shot of the end of life plan worksheet of FIG. 12A.

FIG. 12E is a fifth screen shot of the end of life plan worksheet of FIG. 12A.

FIG. 12F is a sixth screen shot of the end of life plan worksheet of FIG. 12A.

FIG. 12G is a seventh screen shot of the end of life plan worksheet of FIG. 12A.

FIG. 12H is an eighth screen shot of the end of life plan worksheet of FIG. 12A.

FIG. 12I is a ninth screen shot of the end of life plan worksheet of FIG. 12A.

FIG. 12J is a tenth screen shot of the end of life plan worksheet of FIG. 12A.

FIG. 12K is an eleventh screen shot of the end of life plan worksheet of FIG. 12A.

FIG. 12L is a twelfth screen shot of the end of life plan worksheet of FIG. 12A.

FIG. 12M is a thirteenth screen shot of the end of life plan worksheet of FIG. 12A.

FIG. 12N is a fourteenth screen shot of the end of life plan worksheet of FIG. 12A.

FIG. 12O is a fifteenth screen shot of the end of life plan worksheet of FIG. 12A.

FIG. 12P is a sixteenth screen shot of the end of life plan worksheet of FIG. 12A.

FIG. 12Q is a seventeenth screen shot of the end of life plan worksheet of FIG. 12A.

FIG. 12R is an eighteenth screen shot of the end of life plan worksheet of FIG. 12A.

FIG. 12S is a nineteenth screen shot of the end of life plan worksheet of FIG. 12A.

FIG. 12T is a twentieth screen shot of the end of life plan worksheet of FIG. 12A.

FIG. 12U is a twenty-first screen shot of the end of life plan worksheet of FIG. 12A.

FIG. 12V is a twenty-second screen shot of the end of life plan worksheet of FIG. 12A.

FIG. 12W is a twenty-third screen shot of the end of life plan worksheet of FIG. 12A.

FIG. 12X is a twenty-fourth screen shot of the end of life plan worksheet of FIG. 12A.

FIG. 12Y is a twenty-fifth screen shot of the end of life plan worksheet of FIG. 12A.

FIG. 12Z is a twenty-sixth screen shot of the end of life plan worksheet of FIG. 12A.

FIG. 12AB is a twenty-eighth screen shot of the end of life plan worksheet of FIG. 12A.

FIG. 13A is a first screen shot of a medical emergency plan worksheet according to an embodiment of the present invention.

FIG. 13B is a second screen shot of the medical emergency plan worksheet of FIG. 13A.

FIG. 13C is a third screen shot of the medical emergency plan worksheet of FIG. 13A.

FIG. 13D is a fourth screen shot of the medical emergency plan worksheet of FIG. 13A.

FIG. 13E is a fifth screen shot of the medical emergency plan worksheet of FIG. 13A.

FIG. 13F is a sixth screen shot of the medical emergency plan worksheet of FIG. 13A.

FIG. 13G is a seventh screen shot of the medical emergency plan worksheet of FIG. 13A.

FIG. 13H is an eighth screen shot of the medical emergency plan worksheet of FIG. 13A.

FIG. 13I is a ninth screen shot of the medical emergency plan worksheet of FIG. 13A.

FIG. 13J is a tenth screen shot of the medical emergency plan worksheet of FIG. 13A.

FIG. 13K is an eleventh screen shot of the medical emergency plan worksheet of FIG. 13A.

FIG. 13L is a twelfth screen shot of the medical emergency plan worksheet of FIG. 13A.

FIG. 13M is a thirteenth screen shot of the medical emergency plan worksheet of FIG. 13A.

FIG. 13N is a fourteenth screen shot of the medical emergency plan worksheet of FIG. 13A.

FIG. 13O is a fifteenth screen shot of the medical emergency plan worksheet of FIG. 13A.

FIG. 13P is a sixteenth screen shot of the medical emergency plan worksheet of FIG. 13A.

FIG. 13Q is a seventeenth screen shot of the medical emergency plan worksheet of FIG. 13A.

FIG. 13R is an eighteenth screen shot of the medical emergency plan worksheet of FIG. 13A.

FIG. 13S is a nineteenth screen shot of the medical emergency plan worksheet of FIG. 13A.

FIG. 13T is a twentieth screen shot of the medical emergency plan worksheet of FIG. 13A.

FIG. 14A is first screen shot of a user public profile page, according to an embodiment of the present invention.

FIG. 14B is a second screen shot of the user public profile page of FIG. 14B.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

With reference to the drawings, and in operation, the present invention provides a digital asset management system 100 allows a user to provide access to digital assets to a third party upon the occurrence of a predetermined event. More specifically, the digital asset management system 100 allows the user to create an account and to establish one or more events. Each event identifies specific digital assets using digital asset access information and includes an event trigger. Further, the digital asset management system 100, in response to receiving an indication that the event trigger has occurred, provides access to the digital asset, using the digital asset access information to the third party.

### Digital Asset Management System—Architecture

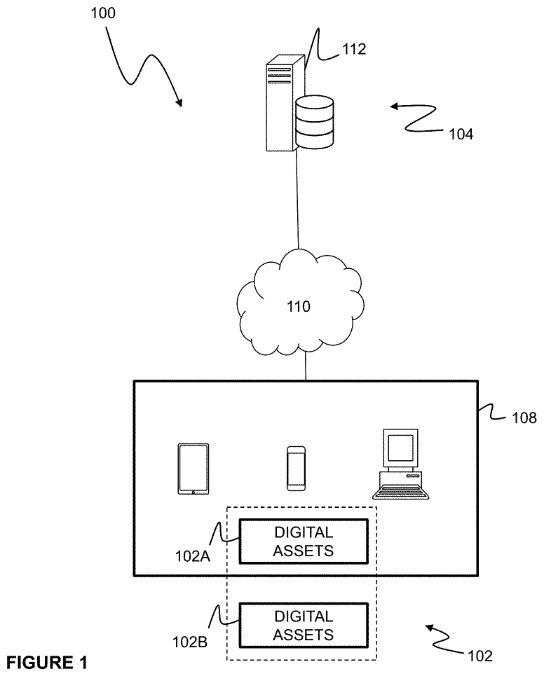

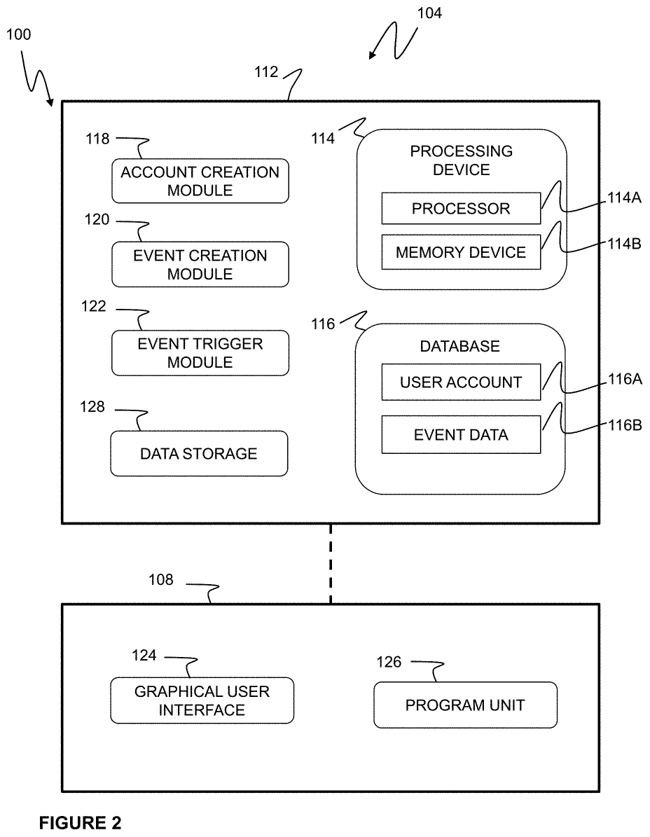

With specific reference to FIGS. 1 and 2, the digital asset management system 100 of the present invention allows a user to create a user account and one or more events. When triggered, an event provides access to specified digital assets to a predetermined third party. The present invention provides a digital asset management system 100 and related methods to transfer ownership and/or access of digital assets 102 to a predetermined third party. In general use, digital asset management system 100 includes a computer system 104 that includes a processing device 114 that is configured (1) to allow a user to create an account and create one or more events and (2) to implement the event upon the occurrence of an event trigger.

Digital assets 102 may be either local digital assets 102A or remote digital assets 102B. The digital assets 102 may include, but are not limited to:
local digital files, such as documents and/or pictures, stored on a local computer system 104 (see below),
access to online accounts, e.g., social media accounts, financial or banking systems, including links, usernames, passwords and other security related information necessary to access such accounts,
access to cloud storage accounts,
digital files, such as documents and/or pictures, stored in a cloud storage system,
digital information, such as account credentials,
digital messages,
digital reference or link to a physical item and other associated digital information, e.g., memorabilia, and
other digital information.

As referenced above, a digital asset 102 may also be, or include, a digital message, e.g., audio, text, picture or audiovisual message, that is recorded specifically (previously and/or by the system during event creation). The digital message may be, for example, personalized message from the user to one or more recipients. The digital message may be a separate digital asset 102 or may be associated with, or part of a any one of the types of digital assets referenced above.

In addition, a digital asset 102 may also be a digital reference to a physical item, such as memorabilia. For example, the user and a recipient may have a shared history with a particular physical item that is in the user's possession. The user may want to pass on the physical item to the recipient. The digital reference or link may include a name and description of the item, where the item is located and/or how to access the item. In addition, the digital reference may include a digital message (see above) that is associated with the physical item. The digital asset 102 may include a story (text, audio, audiovisual or otherwise) associated with the physical item that describes a shared memory or the importance of the item to the user and the reason the user is giving it to the recipient. For example, the user could reference (in a digital reference) a cow bell that was used at the 1984 Olympics kept from a family trip. A parent, as the user, could pass it to their child, as the recipient, that rang it for days on that trip, reminding them of the time they shared together. The physical item may or may not be transferred to the recipient, or referenced in the user's will.

The digital asset management system 100 may provide access to the digital information by:
transferring or sending the digital assets directly to the third party, or
providing the credentials, e.g., logon information, for the external system, such as social media accounts, financial or banking systems, cloud storage accounts to the third party, or,
copying the digital files from the external system or local system to temporary storage and providing access to the temporary storage to the third party.

Referring to FIG. 1, an exemplary environment in which the digital asset management system 100 operates is illustrated. In the illustrated embodiment, the digital asset management system 100 is configured to enable a user to access to the computer system 104 using a user computing device 108.

The user computing device 108 may include any suitable device that enables a user to access and communicate with the digital asset management system 100 including sending and/or receiving information to and from the digital asset management system 100 and displaying information received from the digital asset management system 100 to a user. For example, in one embodiment, the user computing device 108 may include, but is not limited to, a desktop computer, a laptop or notebook computer, a tablet computer, smart phone/tablet computer hybrid, a personal data assistant, a handheld mobile device including a cellular telephone, and the like.

The user computing devices 108 is operably connected to the computer system 104 via a communications network 110. The communications network 110 may be any suitable connection (or combination thereof), including the Internet, file transfer protocol (FTP), an Intranet, LAN, a virtual private network (VPN), cellular networks, Bluetooth and may utilize any suitable or combination of technologies including, but not limited to wired and wireless connections, always on connections, connections made periodically, and connections made as needed.

Communication between the computer system 104 and the user computing device 108 may be facilitated by one or more of the following: a computer program application running on the computer system 104, a computer program application or “app” running on the user computing device 108, a website (hosted by the computer system 104 or an external hosting service (not shown), and a web browser to access/display the website.

The computer system 104 may include a single computer or server computer or multiple computers or server computers. The computer system 104 includes data storage media that is configured to store computer programs and one or more databases for storing user data, such as user accounts and event data. The data storage media may include storage media located directly in one or more computers of the computer system 104, attached data storage, such as external hard drives or NAS storage devices, external cloud storage systems and other suitable devices.

For clarity in discussing the various functions of the computer system 104, multiple functions, devices or units are discussed that are implemented by the computer system 104 and computer program applications. These functions, devices and units may be implemented in multiple different ways such as modules within a single computer, as nodes of a computer system, etc. The functions performed by the computer system 104 (or nodes or modules) may be centralized or distributed in any suitable manner across the computer system 104 and its components, regardless of the location of specific hardware. Furthermore, specific components of the computer system 104 may be referenced using functional terminology in their names. The function terminology is used solely for purposes of naming convention and to distinguish one element from another in the following discussion. Unless otherwise specified, the name of an element conveys no specific functionality to the element or component.

In the illustrated embodiment, the computer system 104 includes a single server computer 112 for simplicity in discussion purposes. However, as noted above, the computer system 104 may include multiple computers or server computers for perform various functions of the digital asset management system 100.

With specific reference to FIG. 2, the server computer 112 may be configured to host a website or provide data to the application or app that is accessible by a user via one or more user computing devices 108. For example, the server computer 112 may retrieve and store a web page associated with one or more websites in response to requests received by the user via the user computing device 108 to allow users to interact with the website and provide access to the digital asset management system 100. In one embodiment, the server computer 112 is configured to generate and display a web page associated with the website in response to requests being received from consumers via corresponding web browsers that are displayed on the user computing devices 12.

Referring to FIG. 2, the in one embodiment, the server computer 112 is communicatively coupled to the user computing device 108 and includes a processing device 114 and a database 116. The processing device 114 executes various programs, and thereby controls components of the digital asset management system 100 according to user instructions received from the user via the user computing device 108. The processing device 114 may include a processor or processors 114A and a memory device 114B, e.g., read only memory (ROM) and random access memory (RAM) for storing processor-executable instructions and that the one or more processors execute. In embodiments where the processing device 114 includes two or more processors 114A, the processors 114A can operate in a parallel or distributed manner. In an example, the processing device 114 may execute and/or implement an account creation module 118, an event creation module 120, and an event trigger module 122 (see below).

The user computing device 108 may include a graphical user interface (GUI) 124 and a program unit 126, which may run a computer-readable program on the user computing device 108.

Additional data storage 128 may also be provided. The data storage 128 may include, but is not limited to, memory device local to one or more computers in the computer system 104 and/or remote storage devices, such as cloud storage. The data storage 128 may be used by the digital asset management system 100 to store user data and/or files, either temporarily or for longer periods (see below).

The database 116 may be located on the server computer 112 or may be located elsewhere and communicatively coupled to the server computer 112. The database 116 may be used to store user and system information, including, but not limited to user account data and event data and/or any suitable information that enables the digital asset management system 100 to function as described herein.

The memory device 114B may be configured to store programs and information in the database 116 and retrieve information from the database 116 that is used by the processor 114A to perform various functions described herein. The memory device 114B may include, but is not limited to, a hard disc drive, an optical disc drive, and/or a flash memory drive. Further, the memory device 114B may be distributed and located at multiple locations.

In one embodiment of the present invention, the memory device 114B may include one or more of the memory devices and/or mass storage devices of one or more of the computing devices or servers. The modules 118, 120, 122 that form part of the digital asset management system 100 are composed of a combination of hardware and software, i.e., the hardware as modified by the applicable software applications. In one embodiment, the units of the present invention are comprised of one of more of the components of one or more of the computing devices or servers, as modified by one or more software applications. The computer programs or software applications define algorithms which, when performed by the processing device 114 of the server computer and/or the program unit 126 of the user computing device 108, comprise the associated module or unit of the digital asset management system 100.

### First Embodiment

### Basic Functionality

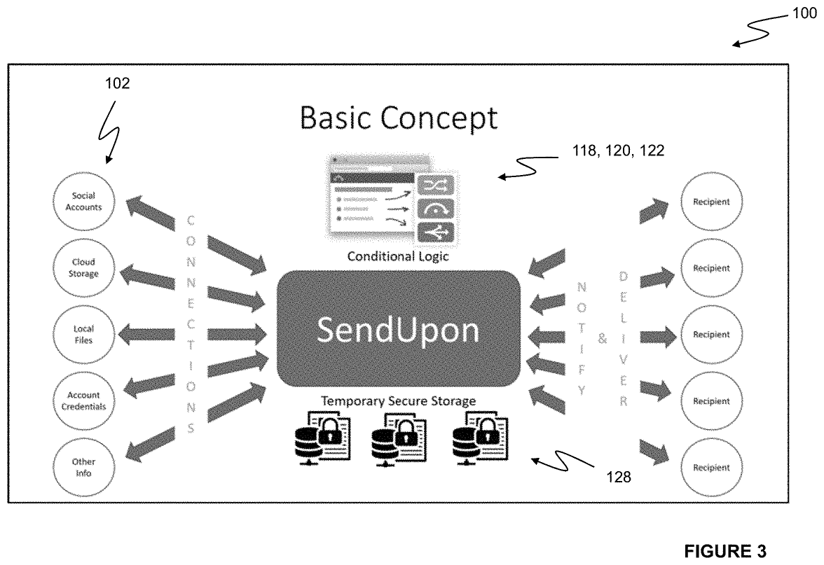

With reference to FIG. 3, operation of, and functionality provided by, one embodiment of the digital asset management system 100 of the present invention is illustrated. In general, the digital asset management system 100 allows users to link different digital assets to defined events within the digital asset management system 100. Each event may include one or more digital assets, an event trigger and one or more recipients.

In one aspect of the invention, the one or more recipients are notified, for example, by email or text messaging, that the event has been created at the time. In other words, the recipient(s) are made aware at the time of creating the event trigger that they will be a recipient of the digital asset in the future. The recipient(s) may be made aware of the nature or type of event trigger, but generally will not have access to the digital asset or its details.

In the illustrated embodiment, the different assets include, but are not limited to social accounts, files stored in cloud storage, local files, account credentials and other information. The digital asset associated with social accounts, may include, where allowed, the account itself or may include the account credentials, for example, the username and password of a social account such as a Facebook account. The digital asset associated with a cloud storage service may include the files stored in the account and/or the account credentials associated with the cloud storage service. A digital asset may include the account credentials for any type of account that may be stored or accessed electronically, including, but not limited to financial or bank accounts, mortgage accounts, insurance accounts, and the like.

Digital assets, whether files such as documents and/or data files containing account details or credentials may be stored within the system either permanently or only temporarily, e.g., after an occurrence of the event trigger (see below). Some embodiments may allow users to upload/store data and files in the data storage 128, e.g., for a fee or based on a subscription model. Alternatively, the data and files may be securely stored temporarily after the occurrence of the event and until they are delivered to the intended recipient.

As shown in FIG. 3, after an event has been defined by a user and the event triggered, the recipient is notified of the event, e.g., the transfer of specific data and/or files from the user to the recipient, and then, depending on the nature of the data and/or files, the data or files are delivered to the recipient.

Account Creation

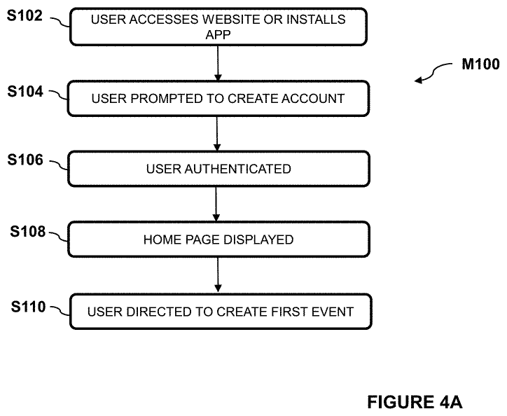

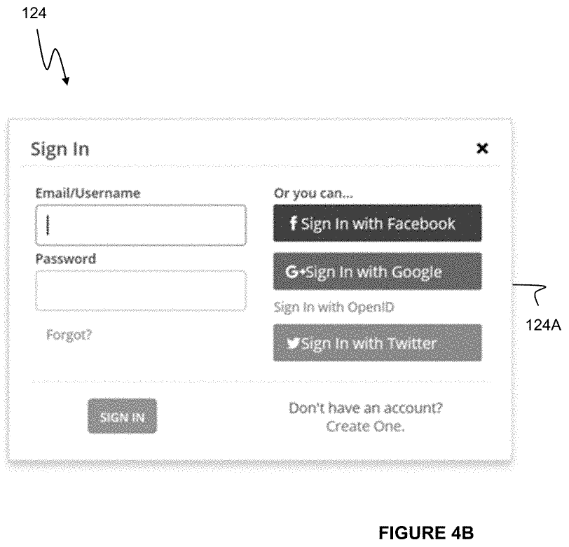

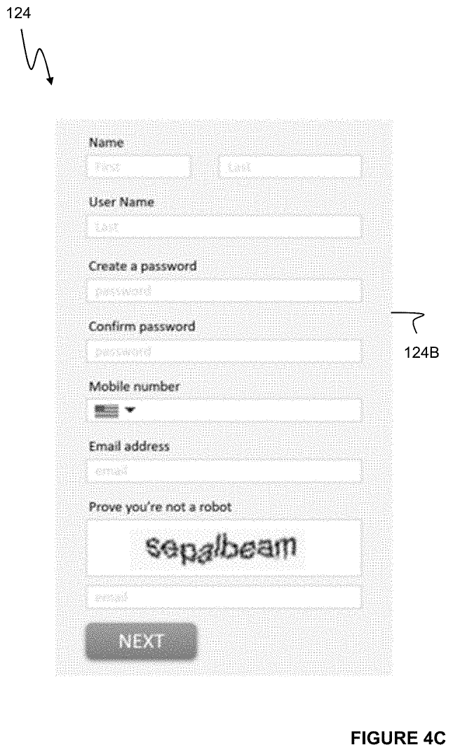

With reference to FIGS. 4A-4C, a first method M100 illustrating operation of the account creation module 118 according to an embodiment of the present invention is shown. In a first step S102, a user accesses a website via a web browser (not shown) using a user computing device 108 or installs an application or app on a user computer device 108.

In a second step S104, the user is prompted to create an account and to enter their desired account details. In a third step S106, the user is authenticated using the entered account details. After the user account details are authenticated, a user account record is created and stored in the database 116.

In a fourth step S108, after the user account has been a create, a home page may be displayed, and the user is directed to create a first event.

With reference to FIG. 4B, an exemplary login dialog 124A is shown. In the illustrated embodiment, the login dialog 124A is displayed using the graphic user interface 124. The login dialog 124A may be displayed when a user accesses the website of the digital asset management system 100 or runs the app or application. The login dialog 124A prompts the user to enter account credentials, e.g., email address/username and password. The user may also login use the login credentials of associated accounts, such as a social network account or email account. In the illustrated embodiment, the user may login with their OpenID credentials or a Facebook or Google (Gmail) account. The login dialog 124 also provides a link to allow the user to create an account if one does not exist.

With reference to FIG. 4C, an exemplary account creation dialog 124B is shown. In the illustrated embodiment, the account creation dialog 124B is displayed using the graphic user interface 124. The account creation dialog 124B allows the user to enter their desired account credentials, include a desired username and password. The details may also be verified using a verification functions such as reCaptcha which verifies that the user is a person and not an automated computer process or “bot”. As discussed above, the digital asset management system 100 may support OpenId and all the user to authenticate using the Facebook, Google or other OpenId credentials. The digital asset management system 100 may store the following in the data base 116: userid, password or OpenID token, first and last name, email address and a telephone number.

Event Creation

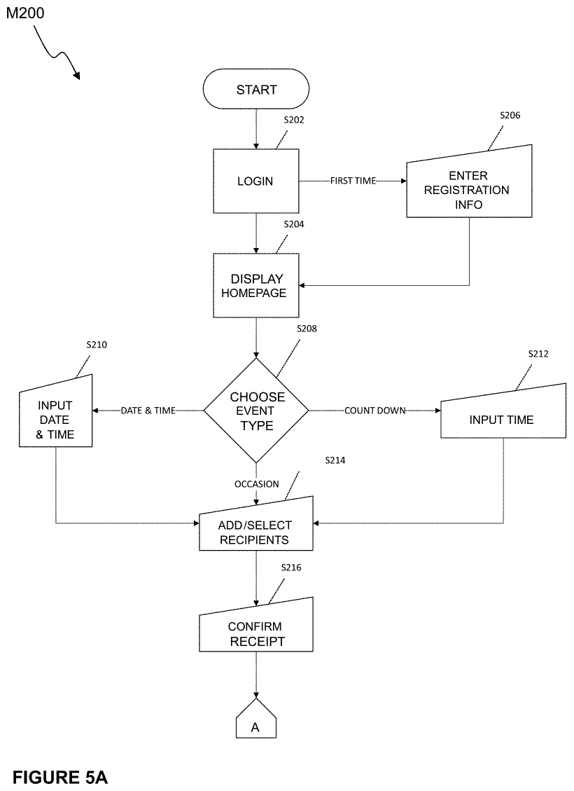

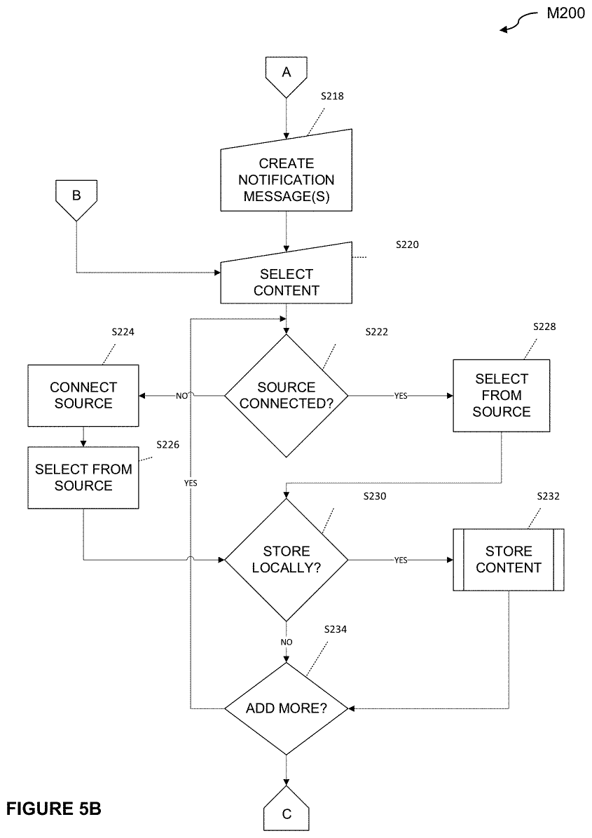

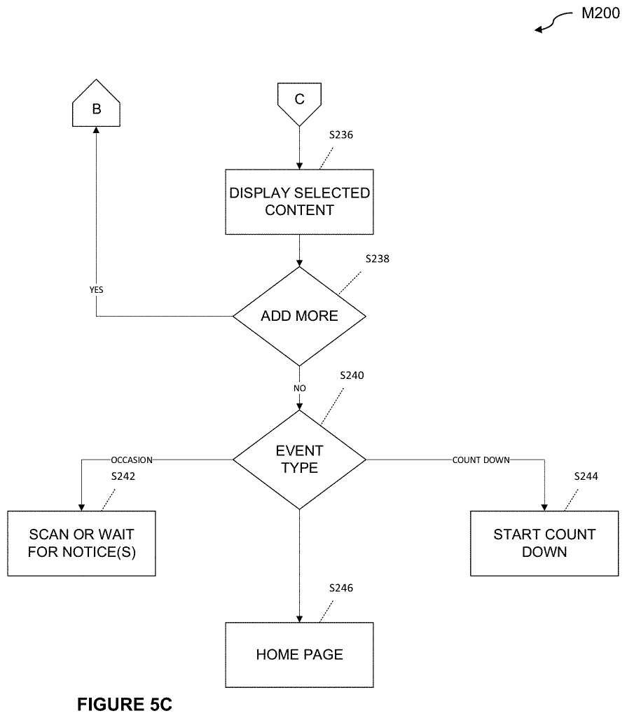

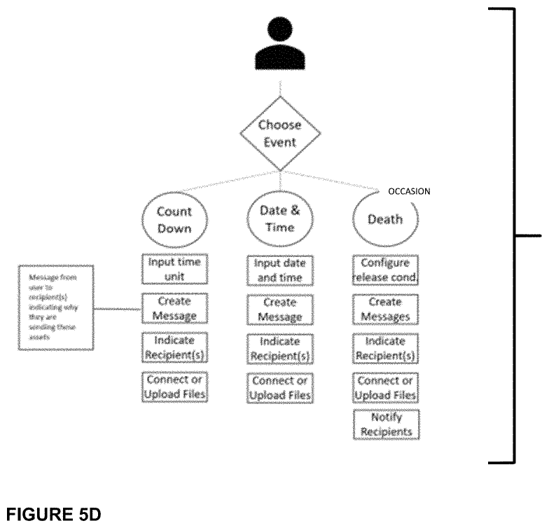

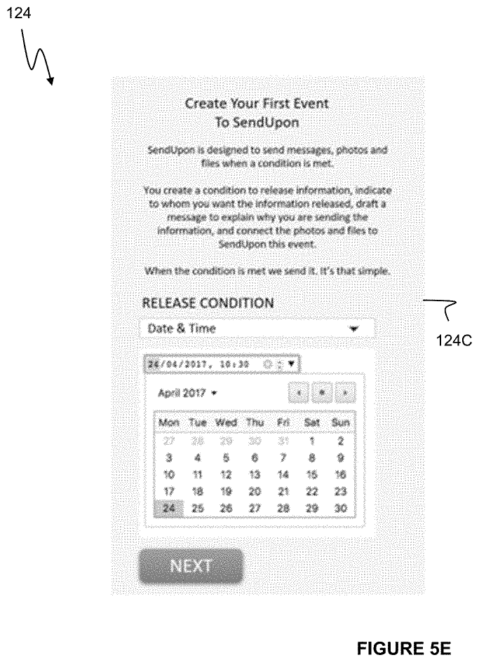

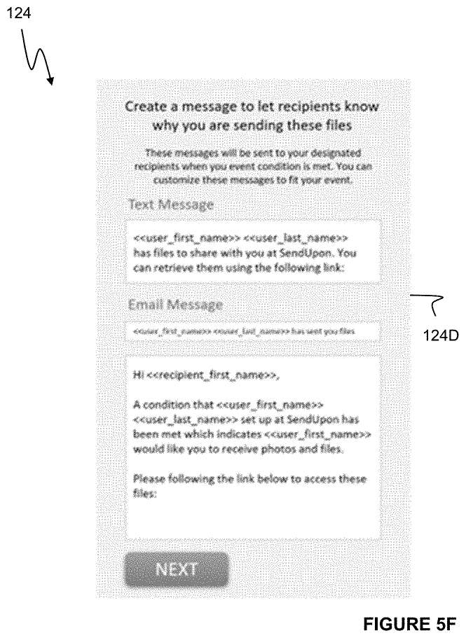

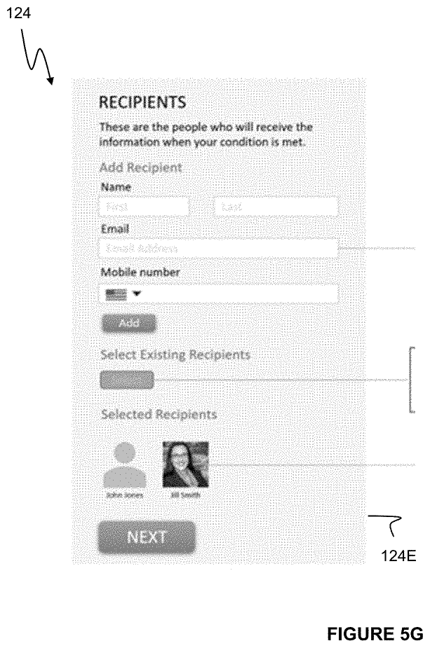

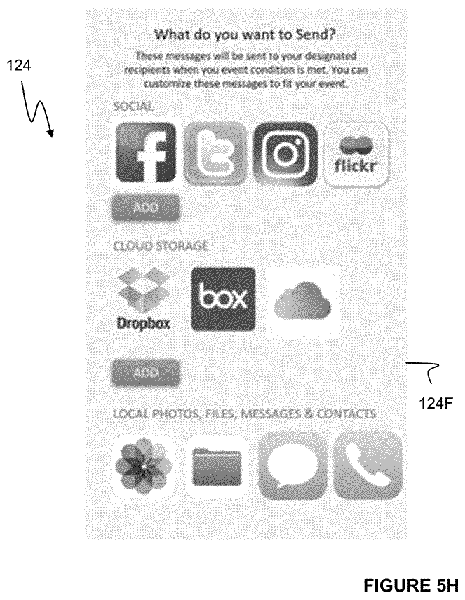

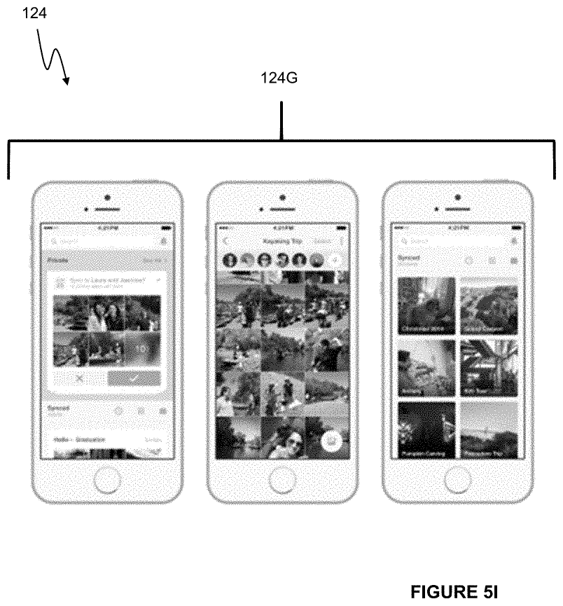

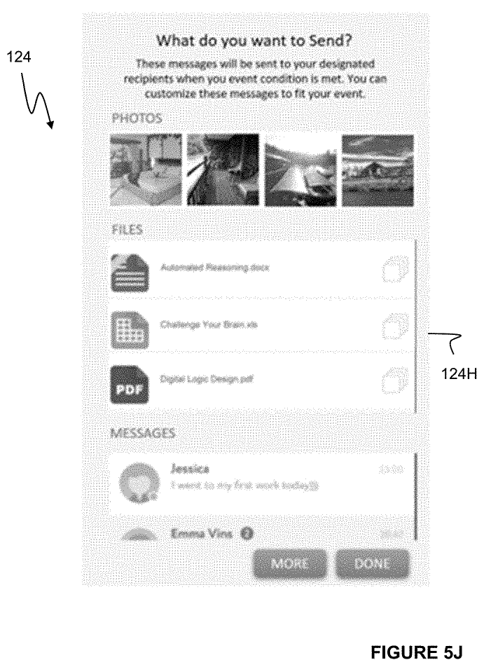

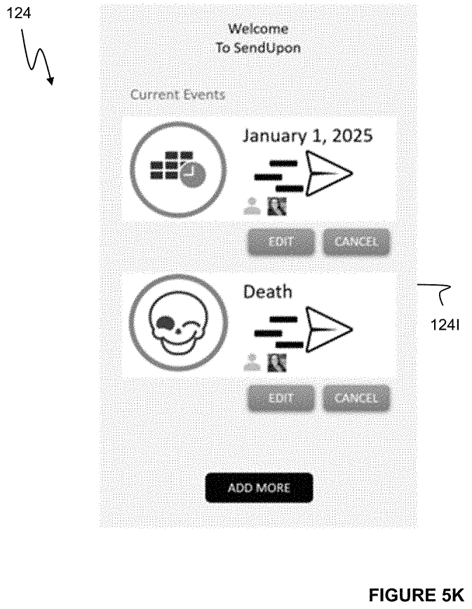

With reference to FIGS. 5A-5K, a second method M200 associated with creation of an event, according to an embodiment of the present invention, is displayed. In one aspect of the invention, each event includes: (1) one or more digital assets, (2) one or more event triggers and (3) one or more recipients In general, after an event is created, the digital asset management system 100 either monitors for, or receives an indication that, the event trigger has occurred. In response to the event trigger having occurred, the digital asset management system 100 notifies the recipient(s) that the digital assets have been transferred or are available.

With specific reference to FIG. 5A in a first step S202, when the user accesses the digital asset management system 100, the login dialog 124A is displayed. As discussed above, the user utilizes the login dialog 124A to either login by entering their account credentials or creating a new account.

If the user has an account and correctly enters their account credentials, then the method M200 proceeds to a second step S202. If the user needs to create a new account, the method M200 proceeds to a third step S206. In the third step S202, the account creation dialog 124B may be shown to facilitate creation of the account (see above). After the account has been successfully created, then the method M200 proceeds to the second step S206.

In the second step S204, a home page is displayed. An exemplary home page 1241 is displayed on the graphic user interface 124 of the user computing device 108 (FIG. 5K). In the exemplary home page 1241, any existing events are displayed, and the user is provided the option to either edit or cancel (delete) each event. The home page 1241 may also provide the user the opportunity to create a new event.

With reference to FIG. 5D, in the illustrated embodiment, the digital asset management system 100 allows the user to create events of one of three types: (1) a countdown event, (2) a date & time event, and (3) a specific occasion event.

A count down event allows the user to specify a duration of time. Once the countdown event has been created. The digital event management system 100 monitors the event and the amount of time that has been passed and once the specification duration of time has passed, initiates the transfer of the specified digital assets (see below).

A date & time event allows the user to specify a specific date & time at which the transfer of the specified digital assets will be transfer. The digital event management system 100 monitors the date & time event and once the specified date and time has been reached, initiates the transfer of the specified digital assets.

A specific occasion event allows the user to specify a specific occasion at which the transfer of the digital asset is to be transferred. For example, the specific occasion may be the death of the user. However, the specific occasion may be any type of occasion, such as a marriage, a divorce, birth of a child, death of another person, a person, e.g., a first-born child, reaching a certain age, and any other specific event. In some instances, the digital asset management system 100 may monitor available resources for the occurrence of the specific and/or may need to be notified of the occurrence of the event (see below).

Returning FIG. 5A, in a fourth step S208, the user selects the type of event to be created. As shown in FIG. 5D, the general process for each type of event is as follows:
Count Down: (1) input time, (2) create message, (3) indicate recipient(s), and (4) connect or upload digital assets or files;
Date & Time: (1) input time, (2) create message, (3) indicate recipient(s), and (4) connect or upload digital assets or files; and,
Occasion: (1) configure release condition, (2) create message, (3) indicate recipient(s), (4) connect or upload digital assets or files, and (5) notify recipients.

With reference to FIG. 5E, an event creation dialog 124C may be displayed by the digital asset management system 100. In the illustrated embodiment the event creation dialog 124C may include a drop-down list which is pre-populated with the types of event available.

If the user selected a date & time event, the method M200 proceeds to a fifth step S210. In the fifth step S210, the user is prompted to enter a date and time at which the transfer of the specified digital assets is to occur (see FIG. 5E). Then, the method M200 proceeds to a seventh step S214.

If the user selected a countdown event, the method M200 proceeds to a sixth step S212. In the sixth step S212, the user is prompted to enter a duration of time after which the transfer of the specified digital asset(s) is to be transferred. Then, the method M200 proceeds to the seventh step S214.

If the user selected an occasion event, then method M200 proceeds to the seventh step S214.

In the seventh step S214, the user is prompted to enter one or more recipients. As shown in FIG. 5G, an exemplary add recipient dialog 124E may be displayed on the graphic user interface 124. In the exemplary add recipient dialog 124E allows the user to enter the name and email and mobile number of a desired recipient. If a recipient is already in the system 100, e.g., in another event, the add recipient dialog 124E allows the user to select without adding in the recipient details again. The add recipient dialog 124E allows the user to add more than one recipient. Added recipients may be shown within the add recipient dialog 124E. The method M200 then proceeds to an eighth step S216.

In the eighth step S216, the digital asset management system 100 verifies the recipient(s) details. For example, the digital asset management system 100 may send each recipient a message, e.g., text or email message, asking the recipient to confirm receipt. The digital asset management system 100 may allow the user to bypass the verification step. The method M200 then proceeds to a ninth step S218.

In the ninth step S218, the user is prompted to create a notification message to be sent to the recipient(s) upon the event being triggered. With reference to FIG. 5F, an exemplary notification creation dialog 124D is shown. The digital asset management system 100 may allow the user to create a notification message to be sent to (1) individual recipients, (2) all recipients, and/or (3) multiple recipients. The exemplary notification creation dialog 124D may be pre-populated with a standard message which the user may edit. The method M200 then proceeds to a tenth step S220.

In the tenth step S220, the user is prompted to select the content or digital assets to send to the recipients when the event trigger (see below) has occurred. Once the content or digital assets have been selected, the method M200 connects the digital asset management system 100 to the content.

The user can connect to other cloud applications through an authentication process, such as OAuth, available from Okta, Inc. This process involves selecting the application, e.g., Facebook, Dropbox, etc., authenticating application to application and authorizing the sharing of information.

Returning to FIG. 5B, once the user selects a source of files in the tenth step S220, in an eleventh step S222, if the source of content is connected, then the method M200 proceeds to a twelfth step S224. In the twelfth step S224 the source is connected, using an authentication process. The method M200 then proceeds to the thirteenth step S226. In the thirteenth step S226, the user is allowed to select the files to be included in the event. Then the method M200 proceeds to a fifteenth step S230.

If, in the eleventh step S222, the source of files is already connected, then the method M200 proceeds to a fourteenth step S228. In the fourteenth step S228, the user is allowed to select the files to be included in the event. Then the method M200 proceeds to the fifteenth step S230.

In the fifteenth step S230, the user is provided the option to have the file(s) stored locally at the digital asset management system 100. If the user elects to have the files stored on the digital asset management system 100, then the method proceeds to a sixteenth step S232. Otherwise, the method M200 proceeds to a seventeenth step S234.

In the sixteenth step S232, the files are transferred from the selected source to the digital asset management system 100. For example, the files may be stored in the data storage 128.

With reference to FIG. 5H, in the illustrated embodiment an exemplary add digital asset dialog 124F is shown. In the illustrated embodiment, the exemplary add digital asset dialog 124G allows the user to select a source or type of content. As the application connects to external sources the user indicates which files at those location they wish to be associated with the event. The user will have the option to choose specific files or indicate that all files should be transferred upon the event trigger. If the user chooses all files, a check box may be presented to include future files created at that location. The user can connect to any approved source and also their desktop and mobile device to indicate files for transfer.

The user can rely on the application to application connection or indicate they want the files transferred to the application for storage (see exemplary file selection dialogs 124G and 124H of FIGS. 5I and 5J, respectively). If the user selected all files with the future files checkbox selected the application will check that location on a periodic, e.g., daily, basis for new files to transfer.

Returning to FIG. 5B, is a seventeenth step S234, the user is prompted to attached additional files from the same source. If the user elects to add additional files, the method M200 returns to the eleventh step S222. If the user is finished adding files from the same source, then the method M200 proceeds to an eighteenth step S232. In the eighteenth step S232, the selected content is displayed and the method M200 proceeds to a nineteenth step S238.

In the nineteenth step S238, the user is provided the opportunity to add more files. If the user elects to add additional files, the method M200 returns to the eleventh step S222. If the user is finished adding files, then the user may be sent back to the home page (step S246).

For occasion type events, in a twentieth step S242, for each event, if the event is an occasion type event, the method M200 proceeds to a twenty-first step S232 and if the event is a count-down type event, the method M200 proceeds to a twenty-second step S244.

In the twenty-first step, the method M200 may scan or monitor various sources or wait for notices that a particular event has occurred. For instance, if the occasion is the death of a user (or third party), the digital asset management system 100 may scan obituary records or the national death registry to determine if the user or third party has died. In addition, the user may have provided instructions for another party, such as church or funeral parlor, to provide notification of death.

For count down occasions, in the twenty-second step S244, a start-down counter is initiated.

### Count Down or Date & Time Event Triggers

Generally, when the countdown is completed or the date and time condition is met, the files are transferred from any remote locations to the data storage 128. If the user had previously elected to store the files in the data storage 128, the digital asset management system 100 checks for any new files or modified files and saves the new or modified files in the data storage 128. The digital asset management system 100 then sends out notifications to the recipient(s) with instructions to retrieve the files. Until a recipient replies or retrieves the file(s), the digital asset management system 100 will continue to notify the recipients for a specified time period, e.g., one year, after the event is triggered.

If/when the files are downloaded by all recipients the application process is considered complete. If the recipients don't respond within the specified period, the files are moved to offline storage and the application continues to try to notify the recipients until their contact information, no longer works or a further time period, e.g., three years, passes (whichever comes first).

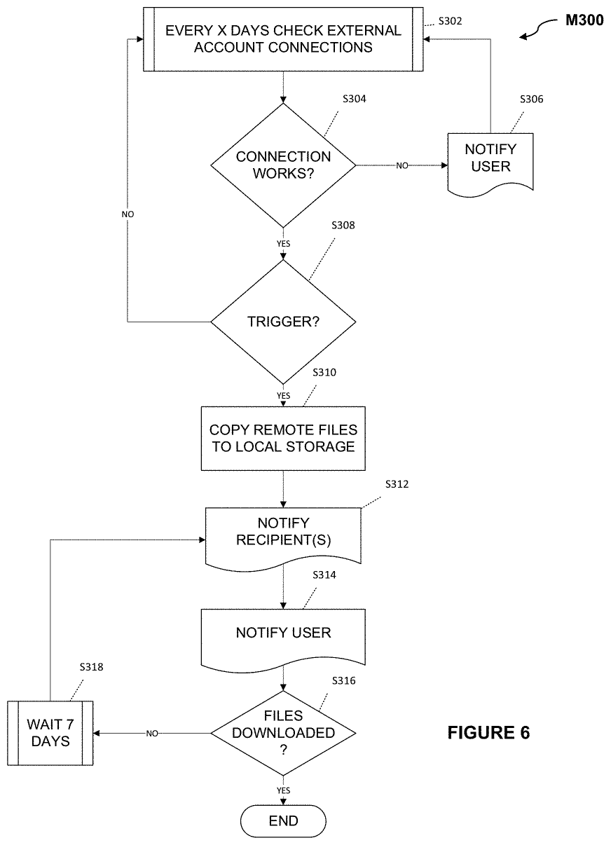

A more detailed method M300, according to an embodiment of the present invention is shown in FIG. 6. In a first step S302, the digital asset management system 100 checks the connections to any external systems, e.g., social accounts or cloud storage systems. This check is performed periodically, i.e., every X days. This period is configurable.

In a second step S304, if the connection to the external system does not work then the method M300 proceeds to a third step S306. In the third step S306, the user is notified that the connection to the external system does not work, providing an opportunity for the user to fix the condition. If the connection works, then the method M300 proceeds to a fourth step S308. In the fourth step S308, if the event trigger has occurred, i.e., date reached or countdown counter reached, then the method M300 proceeds to a fifth step S310. Otherwise, the method M300 returns to the first step S302.

In the fifth step S310, any remote files are copied to the local storage, i.e., data storage 128. If any files are currently stored in the data storage 128, the external system is checked for any updates or new files. Then the method M300 proceeds to a sixth step S312.

In the sixth step S312, the recipients are notified that the event has been triggered and that the files are available, e.g., from the data storage 128 and the method proceeds to a seventh step S314. In the seventh step S314, the user is notified that the event has been triggered and/or the recipient(s) have been notified and/or the files have been made available to the recipient(s) in the data storage 128. The method M300 proceeds to an eighth step S316.

In the eighth step S316, a check is made to determine if the files, in the data storage 128, for example, have been downloaded by the recipient(s). For any recipient that has not downloaded the files stored in the data storage 128, after a period of, e.g., 7, days, (ninth step S318), the method M300 returns to the sixth step S312.

Occasion Event Triggers

Generally, the digital asset management system 100 may trigger an occasion type event by either monitoring an external source or by receiving a notice from another party.

One type of occasion event is an event that transfers digital assets upon the death of the user or a third party. In one embodiment, the digital asset management system 100 may become aware of the death event in one of three ways: (1) obituary found in press source, (2) death notice found in national death registry, or (3) someone presents an official death notice for a user or third party specified in the event.

In one embodiment, the digital asset management system 100 may scan the external sources, e.g., the internet, for obituaries periodically, e.g., every day. If the digital asset management system 100 finds an obituary for a user/third party with a death event trigger, the system 100 will attempt to validate death has occurred. This may be done by initially attempting to contact the user. The application will attempt to contact the user through both email and text message. If the user responds to either message indicating the user or the third party are not dead, the system 100 will ignore the obituary.

If the user cannot be contact after multiple tries the application will then attempt to contact recipients associated with the event. Recipients will be asked to validate death by providing a death certificate or to invalidate death by responding in the negative to the messages.

The digital asset management system 100 may also be presented a death certificate by a recipient, family member of the user, legal fiduciary for the user, or through scanning the National Death Registry (NDR). When the application receives a death certificate it will immediately transfer all files from remote sources to local storage. This is done to ensure those applications do not expire prior to delivery. At the same time the digital asset management system 100 will notify the recipients using the notification message the user programmed.

In one embodiment, the digital asset management system 100 will store files in the data storage 128 for a specified period, e.g., one year, and attempt to notify the recipients periodically, e.g., every thirty days. If no one responds to repeated notifications, the account may be considered abandoned and the files will be archived offline. Offline storage will be kept for a further period, e.g., ten years or until someone claims the files. After the further period, the files will be considered abandoned and may be deleted.

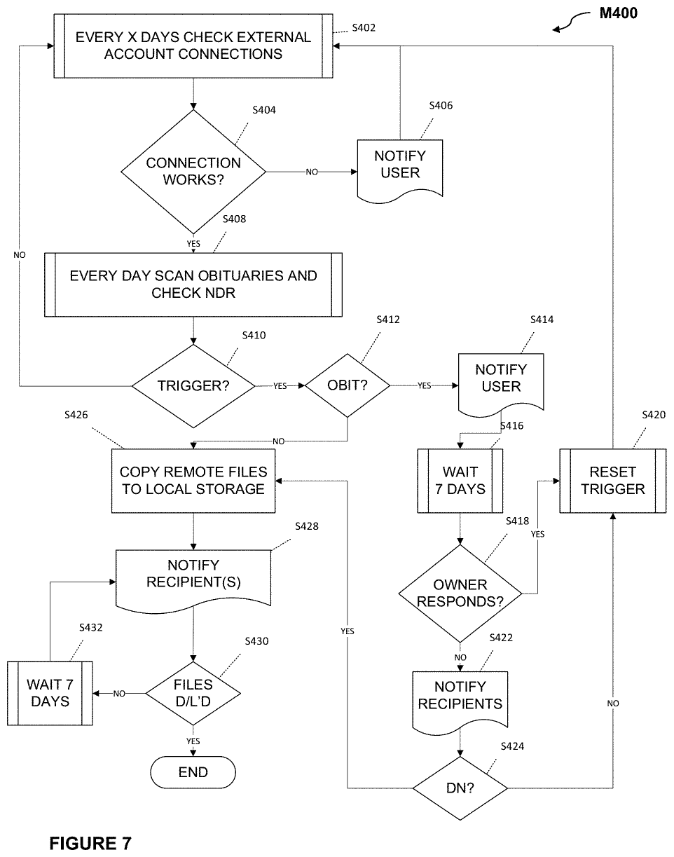

A more detailed method M400, according to an embodiment of the present invention is shown in FIG. 7. In a first step S402, the digital asset management system 100 is configured to check connections to any external systems, e.g., social accounts or cloud storage systems. This check is performed periodically, i.e., every X days. This period is configurable.

In a second step S404, the system method performs logic to determine if a connection actively works or fails. If the connection to the external system does not work, then the method M400 proceeds to a third step S406. In the third step S406, the user is notified that the connection to the external system does not work, providing an opportunity for the user to fix the condition. If the connection works, then the method M400 proceeds to a fourth step S408.

In the fourth step S408, the digital asset management system 100 scans obituaries and checks the National Death Register (NDR) and the method M400 proceeds to a fifth step S410.

In the fifth step S410, if the event trigger has occurred, i.e., the user or specified third party has appeared in the obituaries or the National Death Register (NDR), then the method M400 proceeds to a sixth step S412. It should be noted that the event could also be triggered if the digital asset management system 100 is otherwise notified of the user or specified third party's death, e.g., by a designated or non-designated party.

In the sixth step S412, if the event trigger was an obituary, then the method M400 proceeds to a seventh step S414 to attempt to verify the death. In the seventh step S414, the user is notified of the potential event trigger. After a specified period, e.g., seven days (eighth step S416), the method M400 proceeds to a ninth step S418. If, in the ninth step S418, the user has responded, indicating that the user (or specified other party) has not died, then the method M400 proceeds to a tenth step S420. In the tenth step S420, the event trigger is reset and the method M400 returns to the first step S402.

If, in the ninth step S418, the user has not responded then the method M400 proceeds to an eleventh step S422. In the eleventh step S422, the recipient(s) are notified and confirmation (in the form of a death notice or certificate) is requested.

The method S400 then proceeds to a twelfth step S424. In the twelfth step S424, if the death is not confirmed, then the method proceeds to the tenth step S420. Otherwise, the method proceeds to a thirteenth step S426.

If in the sixth step S412, the event trigger is an appearance of a death notice in the National Death Register (NDR), then the method S400 proceeds directly to the thirteen step S426.

In the thirteenth step S426, any remote files are copied to the local storage, i.e., data storage 128. If any files are currently stored in the data storage 128, the external system is checked for any updates or new files. Then the method M400 proceeds to a fourteenth step S428.

In the fourteenth step S428, the recipients are notified that the event has been triggered and that the files are available, e.g., from the data storage 128 and the method proceeds to a fifteenth step S430. It should be noted that if the occasion is not the death of the user, then the user may also be notified that the files are available in the data storage 128 for the recipient(s) to claim.

In the fifteenth step S430, if the files have been downloaded by the recipient(s), then the method M400 ends. If the files have not been downloaded by all of the recipients, then the method M400 proceeds to a sixteenth step S432. In the sixteenth step, S432, after a specified period, e.g., seven days, the method M400 returns to the fourteenth step S428.

### Second Embodiment

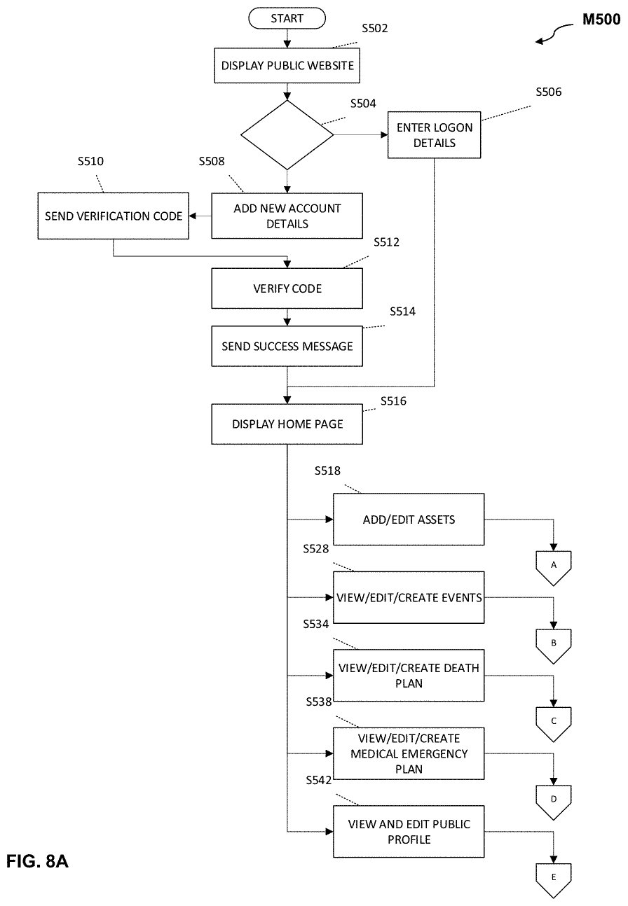

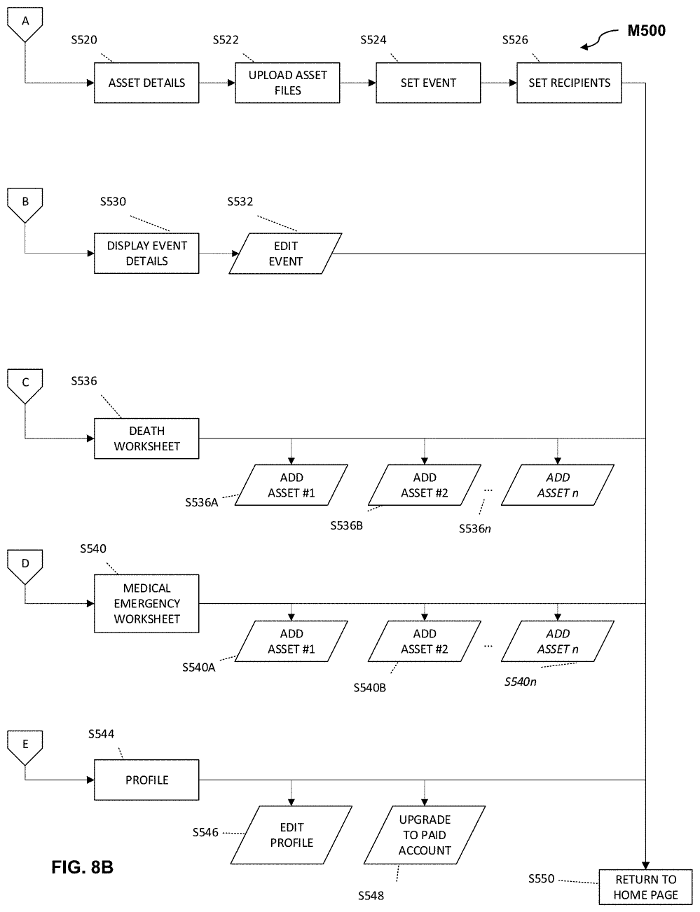

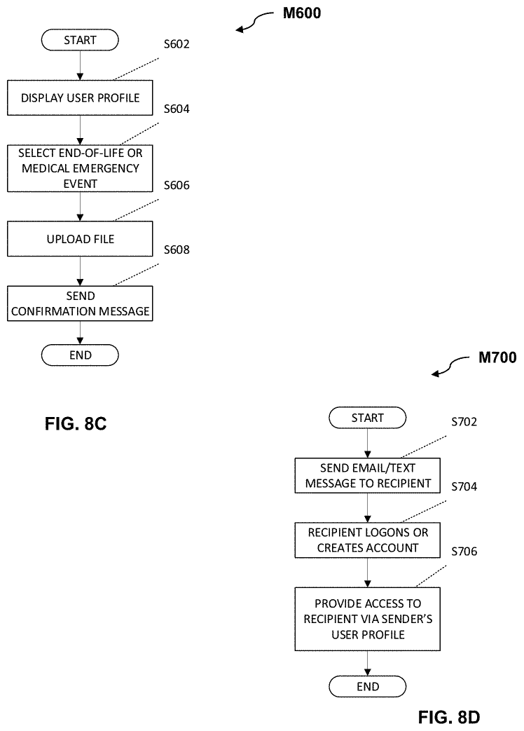

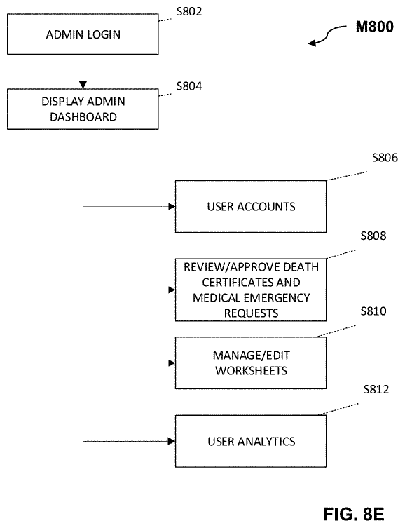

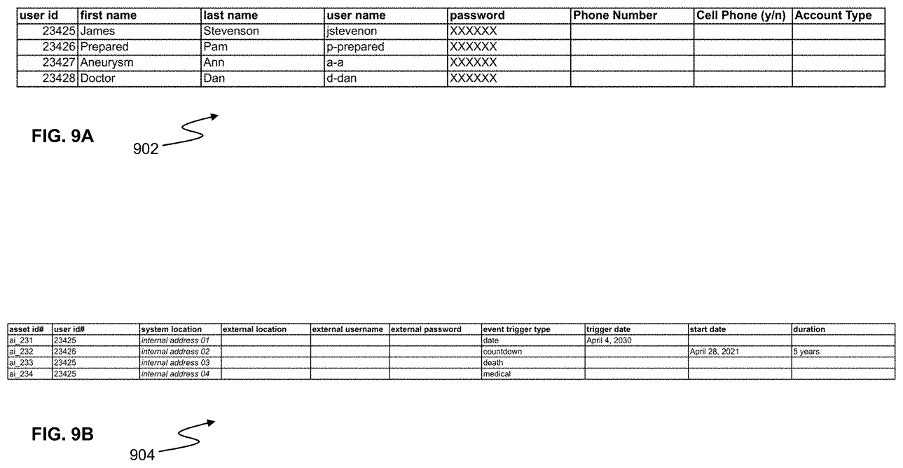

With reference to FIGS. 8A-8B, 9A-9B,10A-10L, 11A-11D, 12A-12Z, 12AB, 13A-13T and 14A-14B, a second embodiment of the present invention is shown.

### Basic Functionality

As discussed in further detail below, after a user creates an account, and/or signs into an existing account via a public website or page 200, the system 10 navigates to a home page 220. Through the home page 220, the system 100 allows the user to create events and/or add digital assets (see below). The home page 220 may also display any events that the user had previously created and/or the events that other users have created for which the user is a recipient.

In the second embodiment, from the home page 220 the user may add digital assets and trigger event(s) for each digital asset and/or individually and/or use a guided process to add digital assets and associated recipients for defined events, e.g., death or a medical emergency. In the illustrated embodiment, guided processes are defined for the death and a medical emergency. The guided processes may be referred to as “end of life” plan and a “medical emergency” plan, respectively. As discussed in further detail below, in the illustrated embodiment, the system 100 may utilize an end of life plan worksheet 240 and a medical emergency plan worksheet 270 to guide the user through recommended or suggested digital assets and allow the user to upload or link the digital asset to the user account and respective plan using the worksheets 240, 270.

Upon initial sign in, the user may see a prompt or invitation on the home page 220 to start a plan or series of events that occur upon their death. For instance, in the illustrated embodiment, if an end plan or a medical emergency plan has not been previously initiated, the user may see the following prompts “Create your End-of-Life Plan” and “Create your Medical Emergency Plan”. Upon selection of one of the prompts, the system 100 will navigate to the respective worksheet 240, 270.

As discussed below, on the end of life worksheet 240, the user will be presented with a series of prompts that encourage them to add documents, notes, and instructions (“Assets”) pertaining to different categories of information that will be important to the user's friends, family, and caregivers after death, i.e., “recipient(s)”. These categories may include healthcare, legal, financial, memorial, and legacy considerations—with items spanning people, locations, documents, accounts, and other. Each category will be populated as assets are added in the above categories). The medical emergency plan worksheet 270 will follow a similar format but will focus specifically on medical information.

Under the memorial category, the following digital assets (which may collectively be referred to as a “funeral plan” may be included:
funeral options, e.g., embalming or cremation;
farewell options, e.g., burial, urn, or ashes spread;
music to be played;
food to be served;
location;
list of people to notify;
other.

In one aspect of the present invention, paid users or subscribers can store files directly within the system 100 in a digital repository. For non-paid users, files may be stored in an external repository or repositories, through third party solutions like Google Drive or Dropbox. In this instance, the digital assets stored in the system provide the necessary information (e.g., a link, username and password) necessary to access the external assets or files.

For each asset a user adds, the user will be prompted to set the event or event trigger for that item, i.e., the event on which the item will be shared with the intended recipient(s), as well as the recipients for that item (i.e., who the item will be shared with once the event occurs).

When setting the event or event triggers, users can choose from a variety of options: they can set a countdown timer or a specific date/time, create a public share link, or specify that the item should only be shared on death or on incapacitation (i.e. ‘Medical Emergency’). Assets added through the end-of-life worksheet 240 will default to the “on death” Event type, and assets added through the medical emergency worksheet 270 will default to the “on Medical Emergency” Event type.

When an event or event triggers occurs, all recipients will receive a notification that tells them how/where to access the assets for which they have been added as a recipient. If the Event was set as On Death or On Medical Emergency, someone must first provide Proof of Death (Death Certificate) or Proof of Medical Emergency.

In one aspect of the present invention, the Proof of Death must be validated. In one embodiment, of the present invention, for example, if a Death Certificate is uploaded, the Death Certificate may be manually verified by a system administrator. Alternatively, the Death Certificate may be automatically verified, for example, by comparison against an external database of death records, such as the National Death Registry (NDR).

In another aspect of the present invention, the Proof of Medical Emergency must be validated. In general, the Proof of Medical Emergency is validated by confirming that the Proof of Medical Emergency originated from a licensed medical professional to avoid, e.g., a violation of the Health Insurance Portability and Accountability Act of 1996 (HIPAA).

In addition to the Death Certificate and Proof of Medical Emergency, the system 100 will capture other details, including from whom (organization and/or person) the request or proof originated for audit purposes.

In one aspect of the present invention, the Proof of In one and the Platform Admin must manually verify that it is authentic. Anyone can upload Proof of Death or Proof of Medical Emergency by visiting the user's user public profile (see below).

With reference to FIG. 8A-8B, a method M500 associated with the second embodiment, is shown. In a first step S502, the user initiates a web application or navigates to the public website 200.

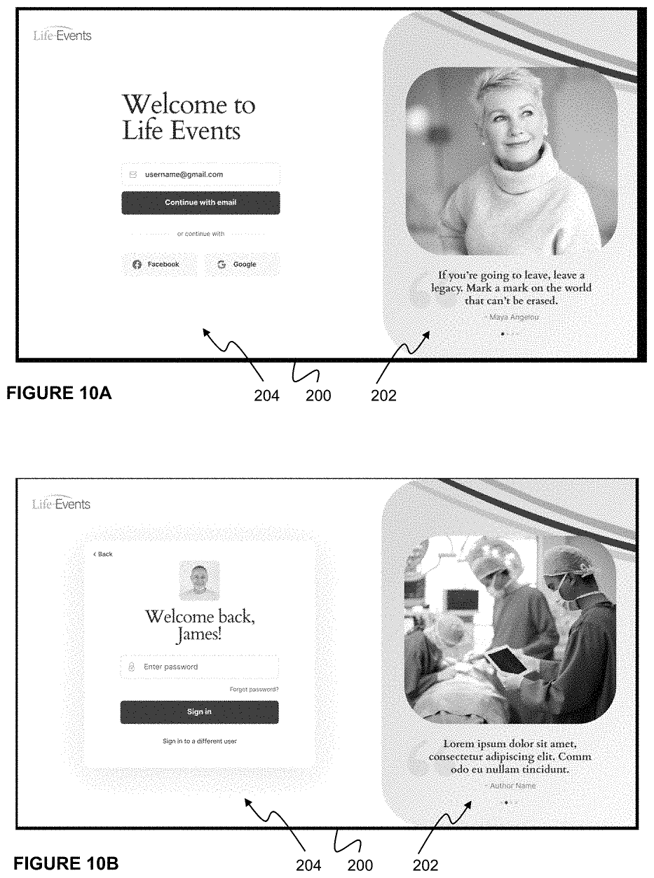

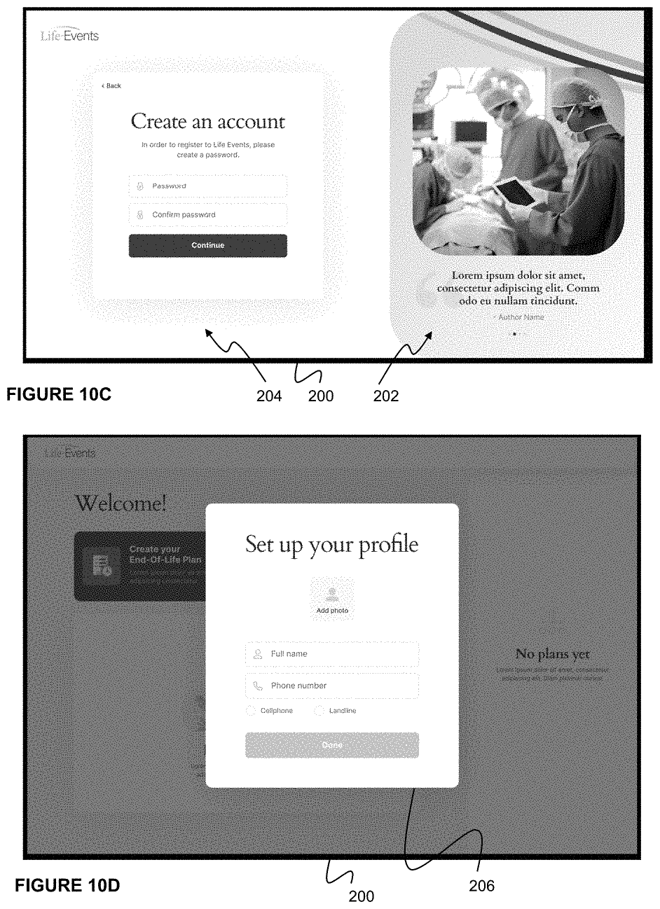

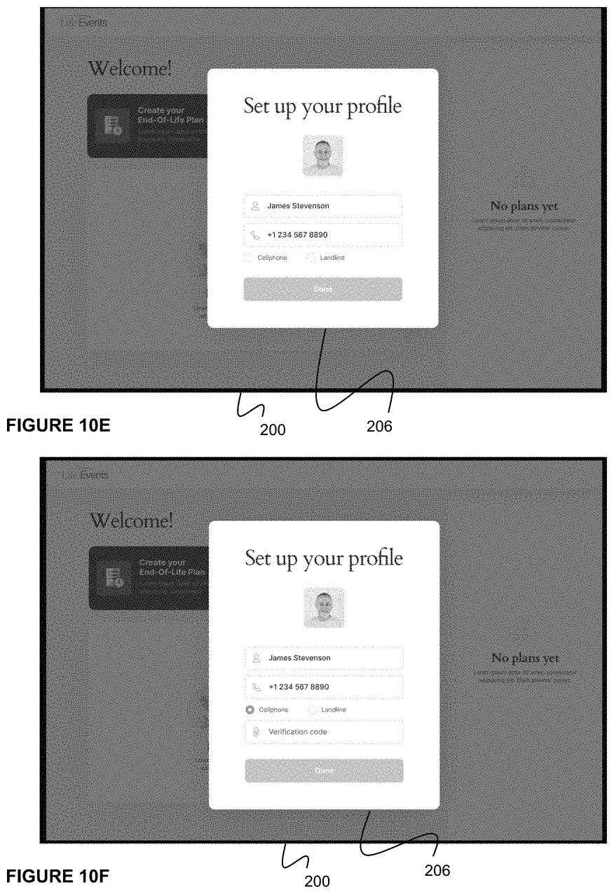

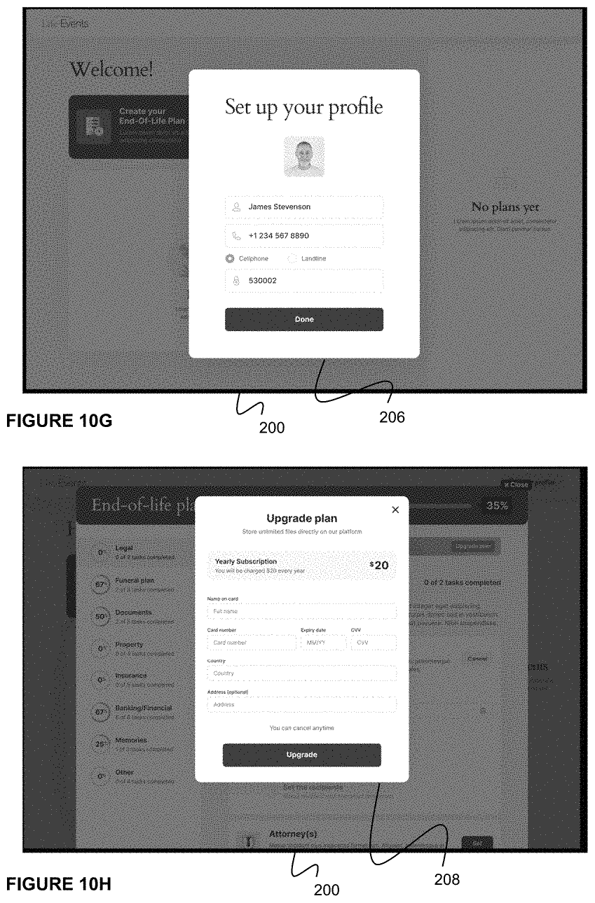

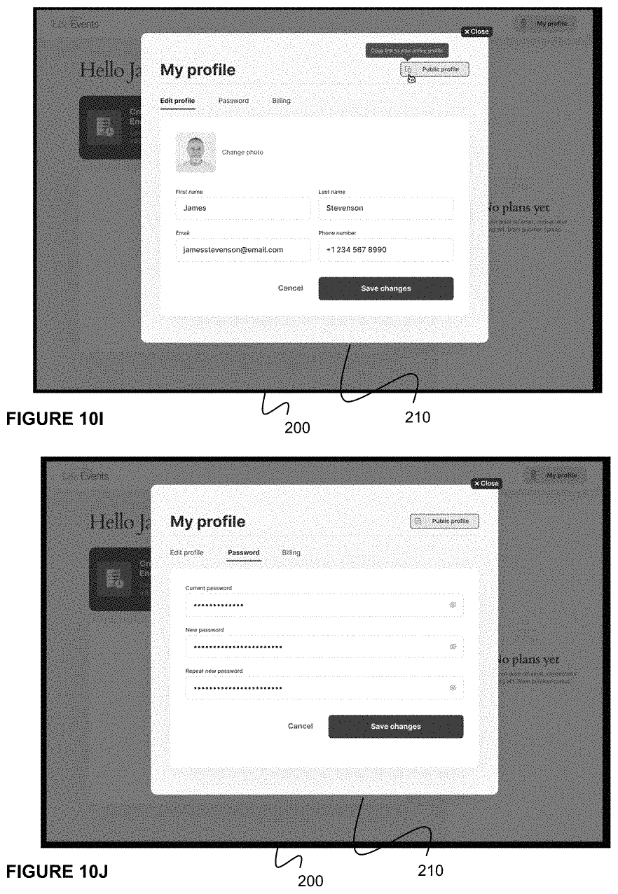

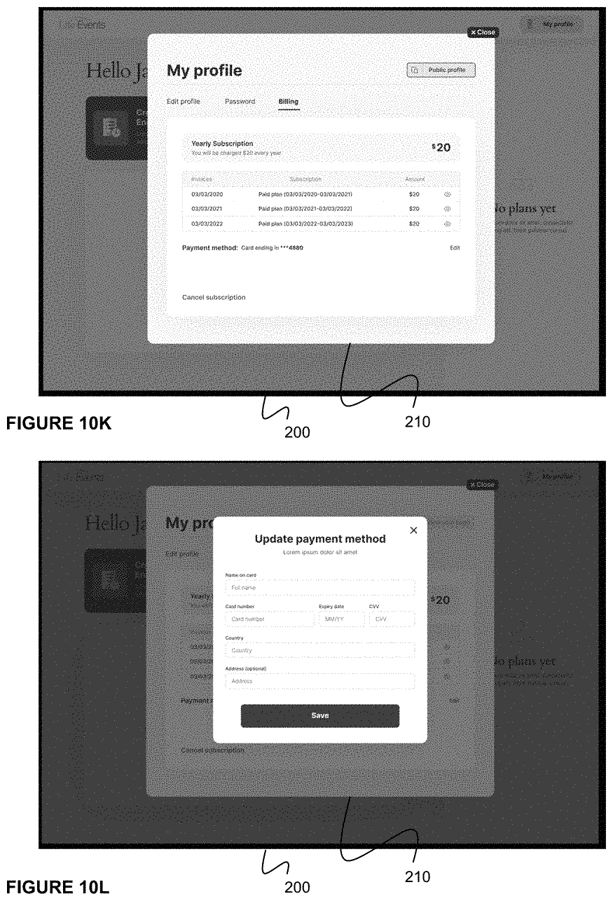

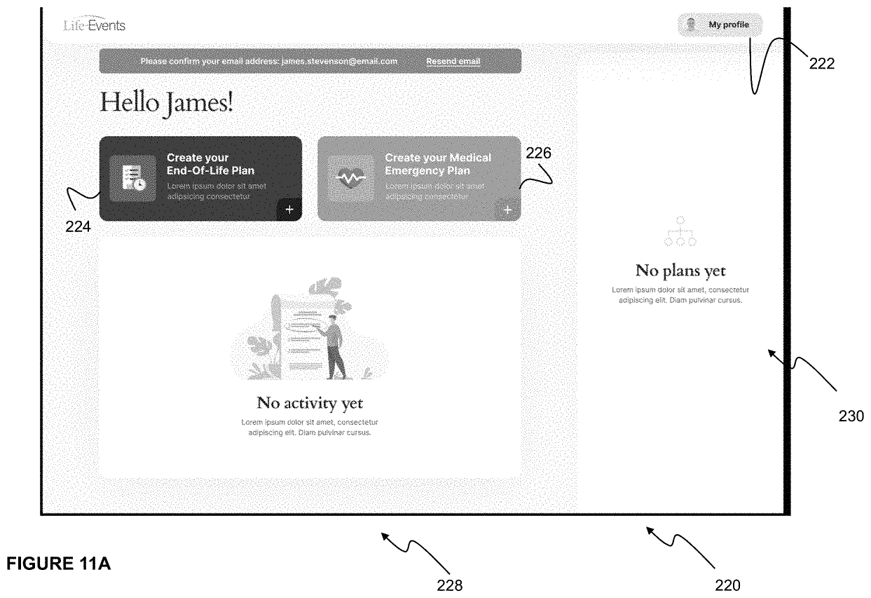

An exemplary public website 200 is shown in FIGS. 10A-10L. The public website 200 allows a user to create a user account and/or to logon to an existing account. In one aspect of the present invention, each user account may be categorized as one of two or more account types tiers which have different cost structures. Each tier may have associated features. For example, in one embodiment, the system provides first and second tiers. In the first tier, which may be free, digital assets in the form of certain digital files, are not stored in the internal repository. Rather, in the first tiers, such digital files must be stored in external repositories. In the second tier, digital files may be stored in an internal repository located within the system 100. The second tier may require a paid subscription.

In a second step S504, the user is presented with an option to either create an account or to logon to an existing account. An exemplary public website 200 is shown. On the right side 202, messages, e.g., inspirational messages or marketing materials, may be displayed to the user. On the left side 204 of the public website 200, the user is presented options to logon or create an account. In the illustrated embodiment, the user may logon or create a new account by entering their email address or use an existing third party account, e.g., a Facebook account or a Google account. If the user selects a third party account, a dialog (not shown) is shown that allows the user to logon into the third party account.

Once the user has entered their email address or logged onto the third party account, the system 100 determines if the user has an account. If the user has an account, the method M500 proceeds to a third step S506. In the third step S506, the user is prompted to enter their password (see FIG. 10B). Once the user has successfully logged on by entering their password, the method M500 proceeds to an eighth step S5S16 in which the home page 220 is displayed (see below).

If, in the second step S504, the user does not have an existing account, the method M500 proceeds to a fourth step S508. In the fourth step S508, the user is prompted to create a user profile by entering new account details. First, the user is prompted to enter and confirm a new password FIG. 10C. Once the new password has been established, the method M500 displays a new account dialog 206 (FIGS. 10D-10G). The new account dialog 206 allows the user to enter additional account details, for example: full name and phone number (FIGS. 10D-10E). During account creation, the account may be upgraded to the second tier or paid account using an upgrade plan dialog 208 (FIG. 10H). In one aspect of the present invention, the account details are stored in a user account record associated with the user.

With reference to FIG. 9A, a representation of a partial user data table 902, including an account record for a plurality of users is shown. In the illustrated embodiment, each account record has an associated unique user identification number or user ID. With reference to FIG. 9A, data related to digital assets may be stored in a data records of a digital asset data table 902. Each record may have a unique digital asset identification number and include the user identification number of the associated user.

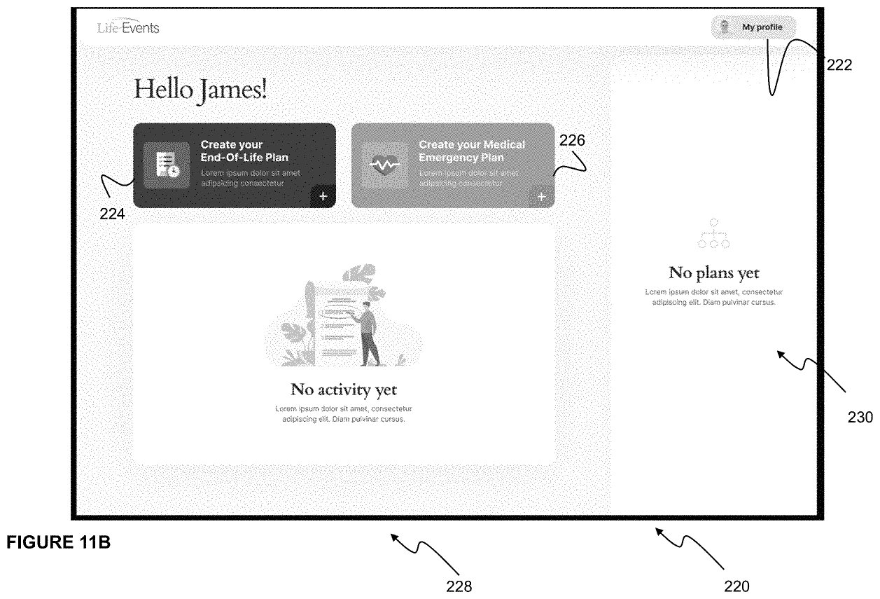

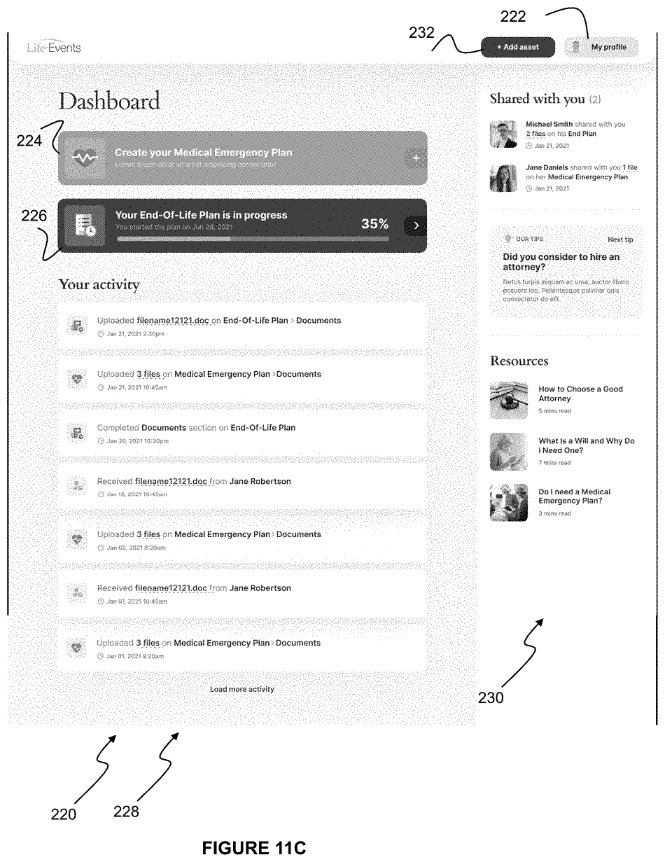

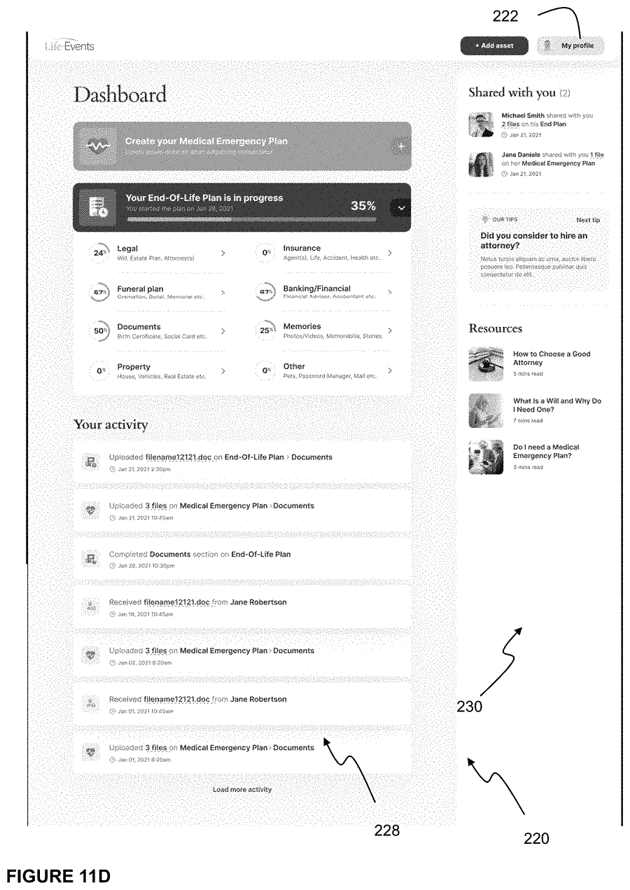

After the new account details have been added in the fourth step S508, the method M500 proceeds to a fifth step S510. In the fifth step S510, the method M500 sends a verification code to the user email address or cell phone. The verification code must be entered in the new account dialog 206 in order to proceed (FIGS. 10F-10G). In a sixth step S512, the entered verification code is verified if the entered code matches the sent code, and the method M500 proceeds to a seventh step S514. In the seventh step S514, a message indicating that verification was successful is displayed and the method M500 proceeds to an eighth step S516. In the eighth step S516, the home page 220 is displayed (FIGS. 11A-11D).

With reference to FIG. 11A, the home page 220 a my profile button 222. Selection of the my profile button 222 allows the user to edit their profile details. If the user selects the my profile button 222, a my profile dialog 210 is displayed. The my profile dialog 210 allows the user to enter or modify their account details (see FIGS. 10K-10J).

Returning to FIGS. 11A-11D, the home page 220 includes an end-of-life button 224 and a medical emergency plan button 226. If the user has not initiated one of the plans, then the respective button 224, 226 will be labeled accordingly, e.g., “Create you End-of-Life Plan” and “Create your Medical Emergency Plan”, as shown. If one of the plans has been started, then the respective button 224, 226 will be labeled to reflect that the plan is in progress and may include an indication of percent completion (see e.g., FIG. 11C).

In the illustrated embodiment, the home page 220 includes a recent activity section 228 and a shared activity section 230. Recent activities performed by the user are listed in the recent activity list 228. Digital assets shared by, or included in the account(s) of, other users that list the current user as a recipient, are listed in the shared activity section 230.

Other information or resources may be displayed in the recent activity section 228 and/or the shared activity section 230, such as, but not limited to, tips, access or links to external resources or recommended third party vendors. In the exemplary screen shots shown in FIGS. 11A and 11B, the user has not performed any recent activities nor has any other user shared any digital asset with the user.

Returning to FIGS. 8A-B, from the home page 220, the user may elect to (a) add or edit digital assets individually; (b) view, edit or create events; (c) view, edit or create a death event plan; (d) view, edit or create a medical emergency plan; and/or view and edit their user profile.

In a ninth step S518, from the home page 220 if the user elects to add or edit an individual digital asset the method M500 proceeds to a tenth step S520. In the tenth step S520, the user may add or edit the assets details. In an eleventh step S522, the user is allowed to upload the digital asset (or provide any access details). In a twelfth step S524, the user is allowed to set the event trigger associated with the digital asset. In a thirteenth step S526, the user is allowed to set or establish the recipients for the digital asset. After the user has finished entering all data for the current digital asset, the method M500 proceeds to a twenty-third step S550. In the twenty-third step S550, the method M500 returns to the home page 220.

Returning to FIGS. 11A-11D, from the home page 220, the user elects to add individual digital assets (outside of the worksheets 240, 270) through selection of an add asset button 232. As with the worksheets 240, 270, once a user adds a digital asset, the will be prompted to set the event or event trigger for that digital asset (see “Setting Events” section below) and set the recipient(s) (see “Setting Recipients” section below). Unlike digital assets added via the worksheets 240, 270, there will be no default event set for digital assets added via the add asset button 232 on the home page 220.

In a fourteenth step S528, from the home page 220 if the user elects to add or edit an individual event the method M500 proceeds to a fifteenth S530. In the fifteenth step S530, the event details are displayed and a user may add or edit the event details in a sixteenth step S532. After the user has finished entering all data for the current event, the method M500 proceeds to the twenty-third step S550. In the twenty-third step S550, the method M500 returns to the home page 220.

In a seventeenth step S534, from the home page 220 is the user elects to add or edit an end-of-life or death event, then the method M500 proceeds to an eighteenth step S536. In the eighteenth step S536, the user is presented with an end-of-life or death worksheet 240. The end-of-life worksheet 240 may guide the user through a list of asset categories. Each category may contain a pre-defined list of digital assets. As explained in further detail below, the method 500 guides the user through the list of categories and allows the user to add digital asset(s) in a series of sub-steps S536A, S536B . . . . S536n. Each sub-step S536A, S536B . . . . S536n may be similar to steps S520, S522, S524, S526.

In another aspect of the present invention, the end-of-life worksheet 240 serves as a guided walkthrough that explains the items or events that the user should prepare and share with different recipients, e.g., family, loved ones, in order to make sure the recipients have everything they need after the user die. The end-of-life worksheet 240 may be broken down by sections or categories with a dropdown of important considerations (with accompanying digital asset(s) listed under each. Under each category, the user may be prompted to upload or enter (via a form) the corresponding digital asset(s). A green check mark may appear next to the items that have been taken care of, and a yellow icon will appear next to the items that still need to be addressed. Next to each top-level section, there will be an icon that indicates its level of completion. An exemplary list of categories or section and possible items and/or digital assets that may be addressed in the end-of-life work sheet is listed below:

Legal
Will & Estate Plan
   Attorney(s)

Funeral Plan (this will be a form)
Cremation/Embalming
Burial or Ashes Spread (check one)
Memorial
   Music
   Food
   Location
   Notifications (friends, family, employer)
   Other

Documents
Birth Certificate
Social Security Card
Tax Returns
Marriage & Divorce Certificates
Important Statements (e.g., utilities)

Property
House
Vehicles
Real Estate
Other

Insurance
Agent(s)
Life
Accident
Health
Homeowners or Renter
Car
Disability
Long-Term Care
Liability

Banking/Financial
Financial Advisor
Accountant
Bank Accounts
Financial Accounts
Pensions
Credit Cards & Loans
Securities
Safety Deposit Box & Safes

Memories
Photos/Videos
Memorabilia
Stories

Other

Pets
Password Manager
Secure Home
Stop Mail

All digital assets added via the end-of-life worksheet 240 with default to the “On Death” Event type (see “Setting Events” section below for more detail). However, the user can override this default to set it to a different event. For each digital asset added via the end-of-life worksheet 240, users will also be prompted to set recipients (see “Setting Recipients” section below for more detail).

In a nineteenth step S538, from the home page 220 is the user elects to add or edit a medical emergency event, then the method M500 proceeds to a twentieth step S540. In the twentieth step S538, the user is presented with a medical event worksheet 270. The medical event worksheet 270 may guide the user through a list of asset categories. Each category contains a pre-defined list of digital assets. As explained in further detail below, the method 500 guides the user through the list of categories and allows the user to add digital asset(s) in a series of sub-steps S536A, S536B . . . S536n. Each sub-step S536A, S536B . . . S536n may be similar to steps S520, S522, S524, S526.

In one aspect of the present invention, the medical event worksheet 270 serves as a guided walkthrough that explains, to the user, the items or digital assets that may important for medical professionals and/or family to have access, e.g., a detailed record of your medical history (should the user become incapacitated and are unable to provide for themselves). If triggered, the medical emergency event will allow medical professionals to request these documents after proving that the documents are needed. Like the end-of-life worksheet 270, the medical emergency worksheet 270 may be broken down by sections or category, with a dropdown of important considerations listed under each. Under each category, the user will be prompted to upload or enter (via a form) the corresponding digital asset. A green check mark will appear next to the items that have been taken care of, and a yellow icon will appear next to the items that still need to be addressed. Next to each top-level section, there will be an icon that indicates its level of completion. An exemplary list of categories or section and possible items and/or digital assets that may be addressed in the medical event worksheet 270 is listed below:

Medical History

Medications

Advance Directives
Medical POA
Living Will
POLST (Physicians Orders for Life Sustaining Treatment)
DNR (Do Not Resuscitate Order)
Organ Donation

Contacts

All digital assets added via the medical emergency worksheet 270 with default to the “On Medical Emergency” Event type (see “Setting Events” section below for more detail). However, the user can override this default to set it to a different event type. For each digital asset added via the medical emergency worksheet 270, users will not be able to set recipient(s). In another aspect of the present invention, digital assets assigned as, or defined by, a medical event may only allow medical professionals (who are not identified in advance) to request such digital asset(s).

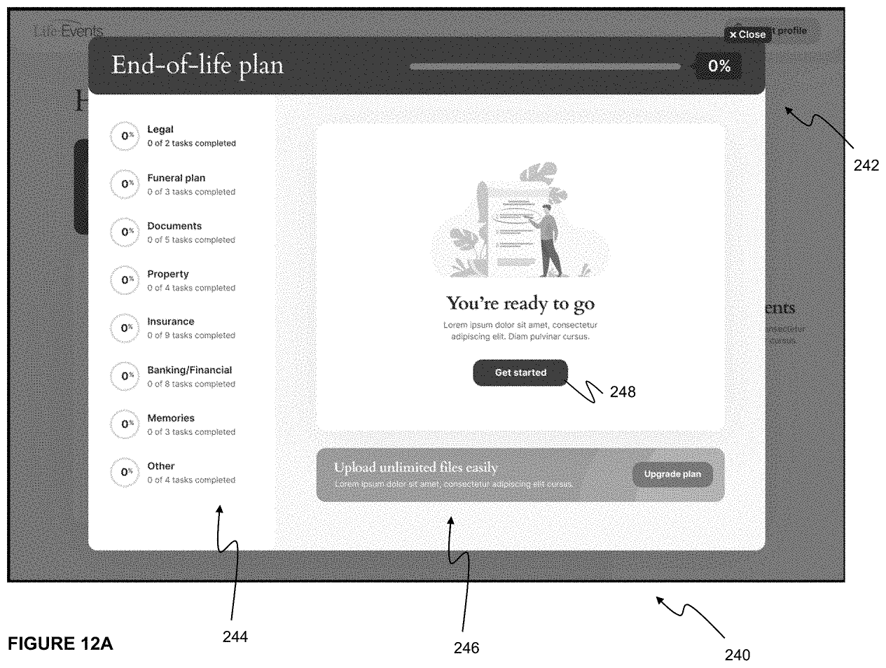

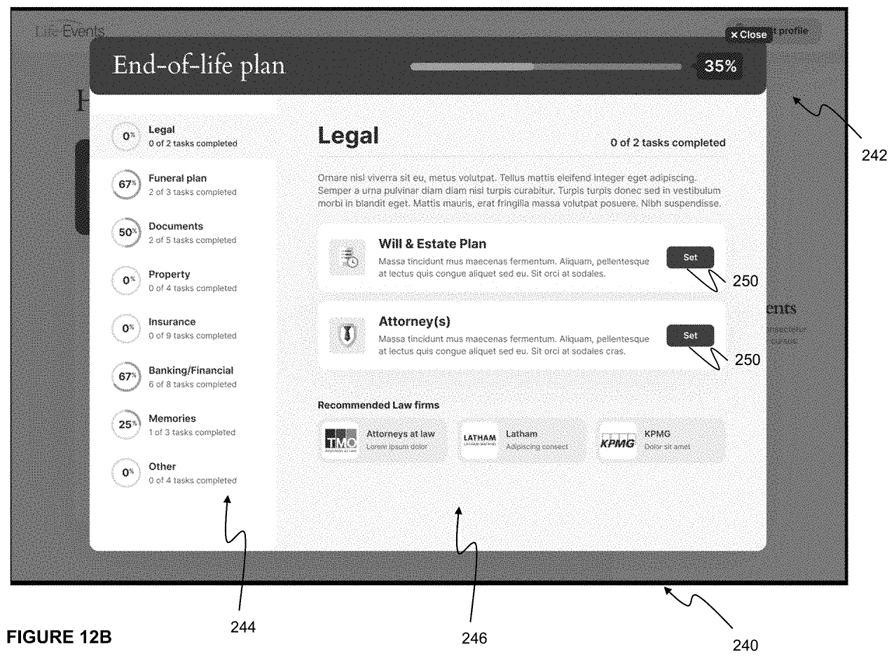

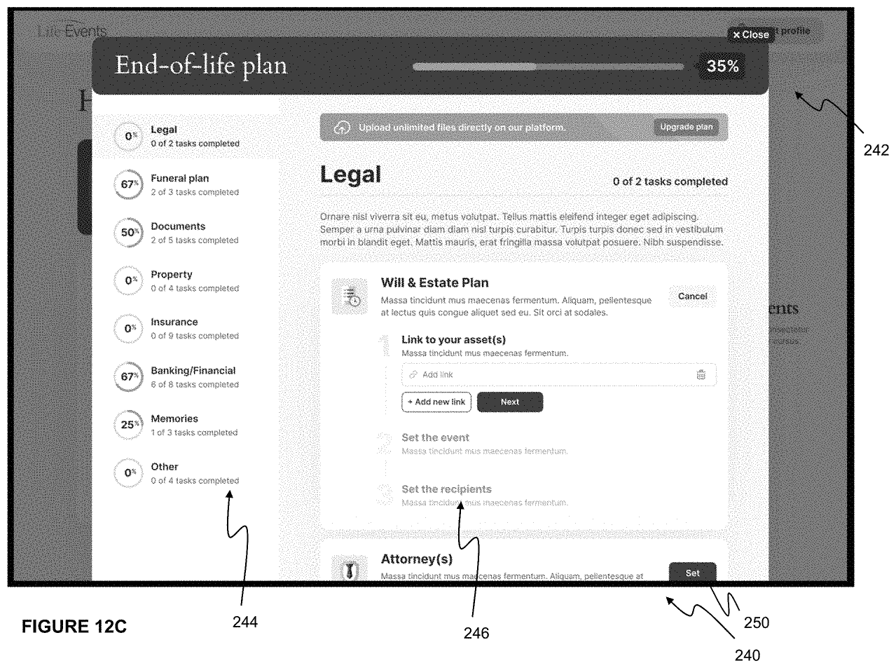

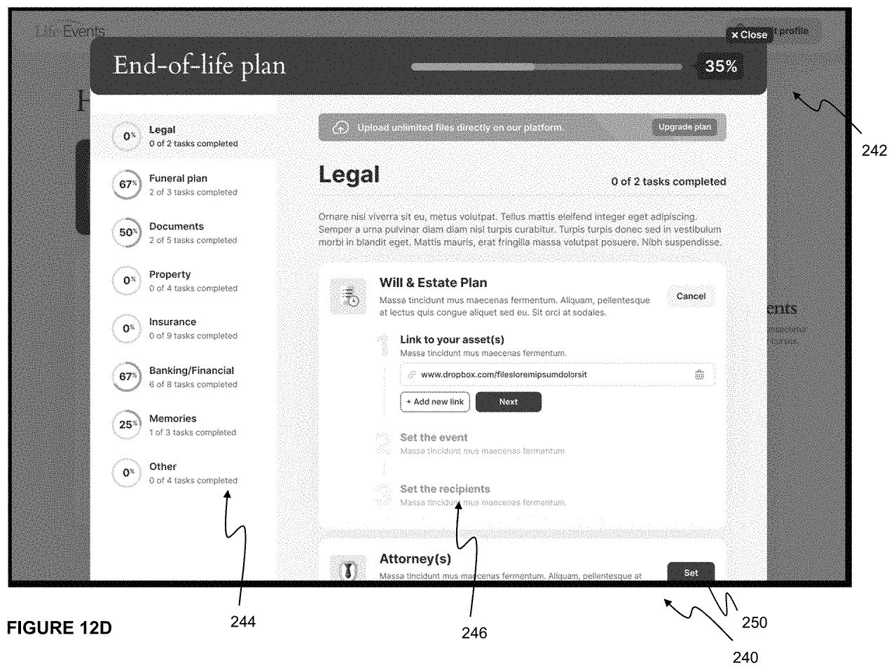

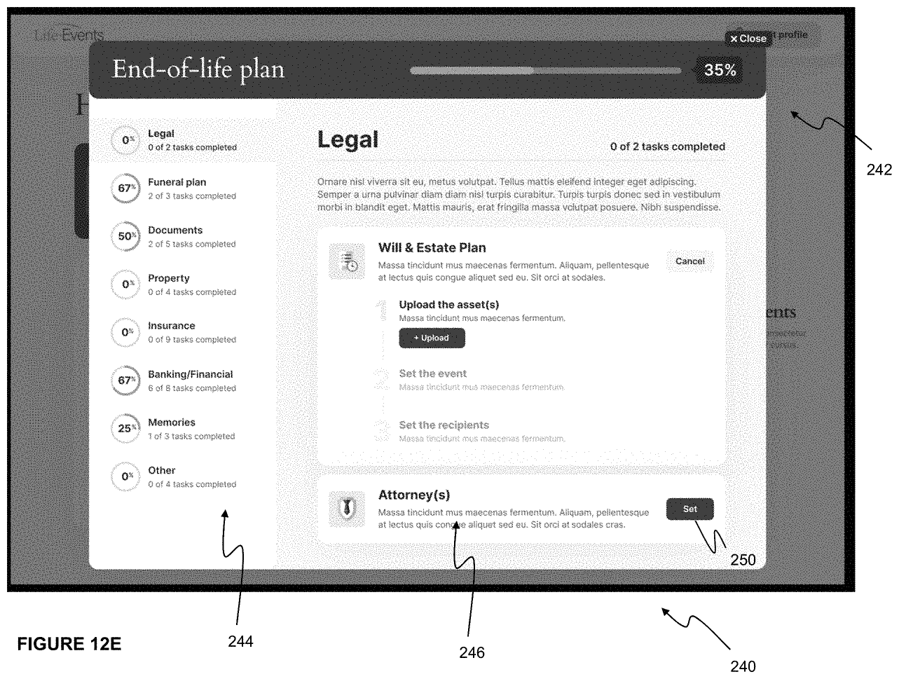

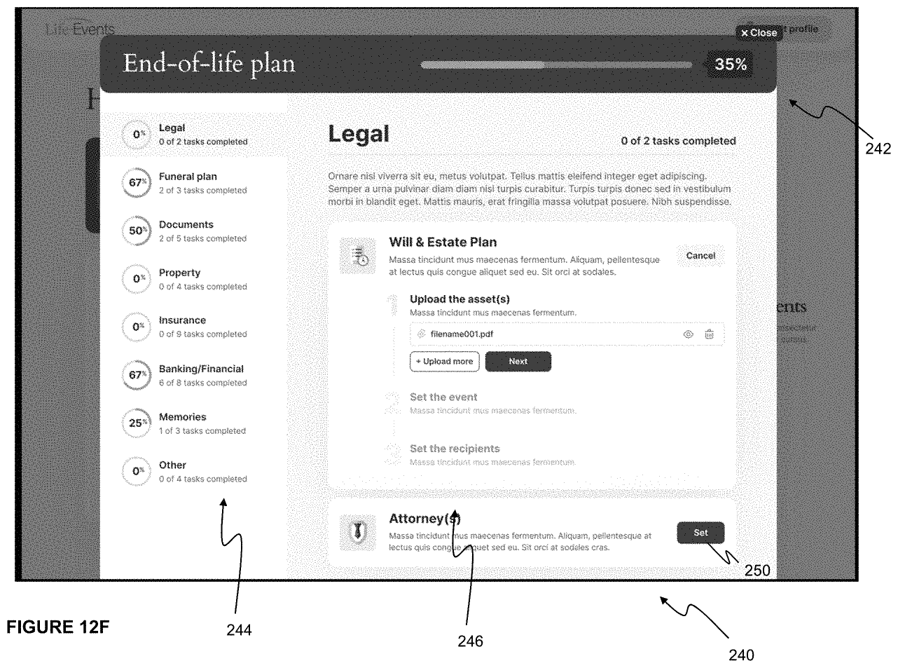

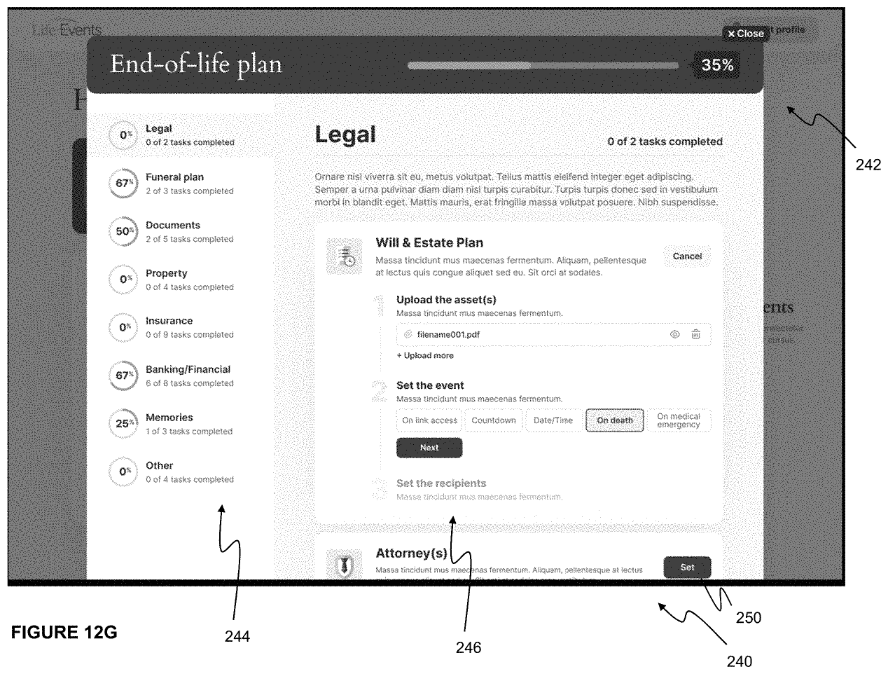

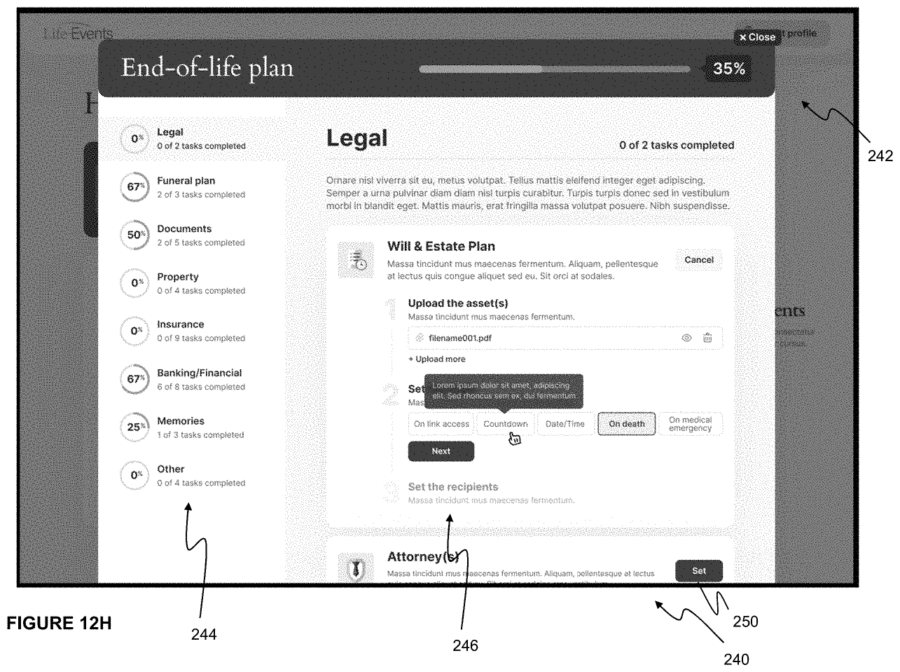

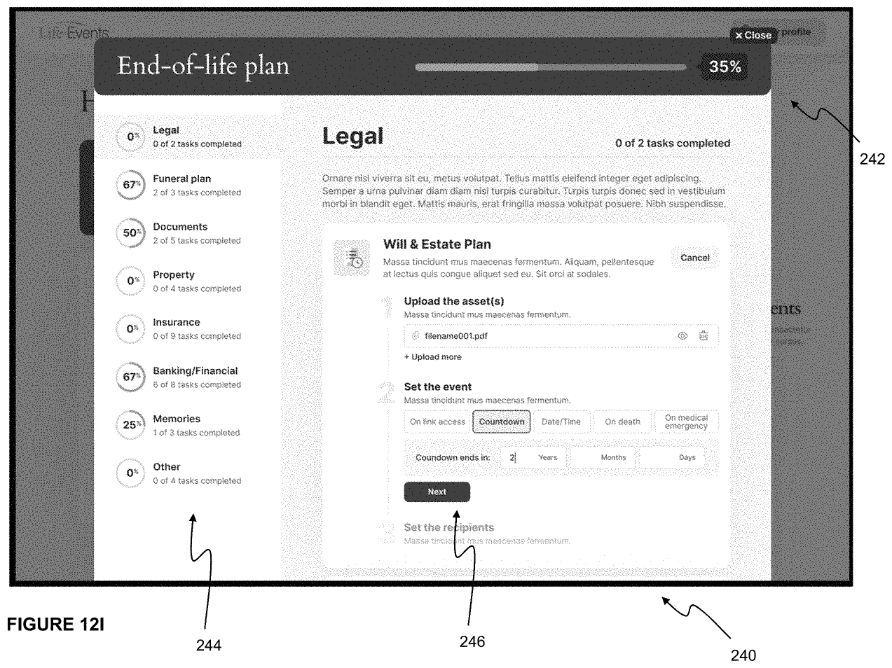

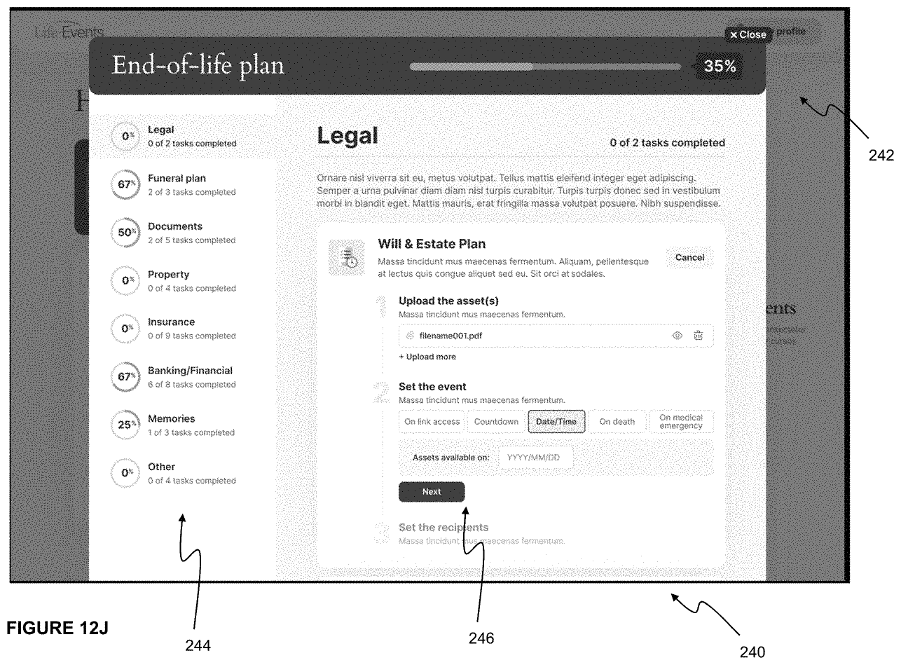

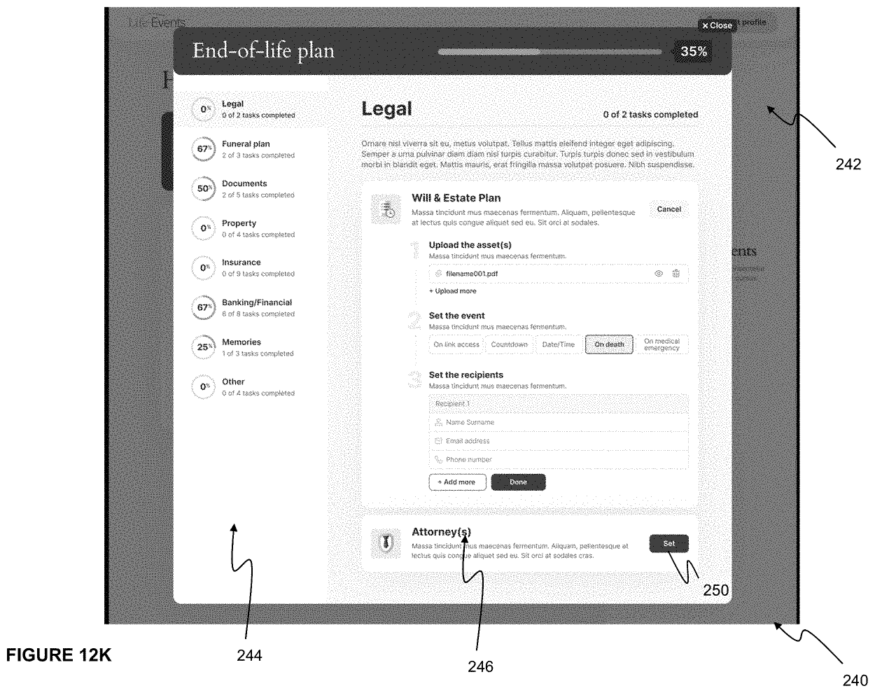

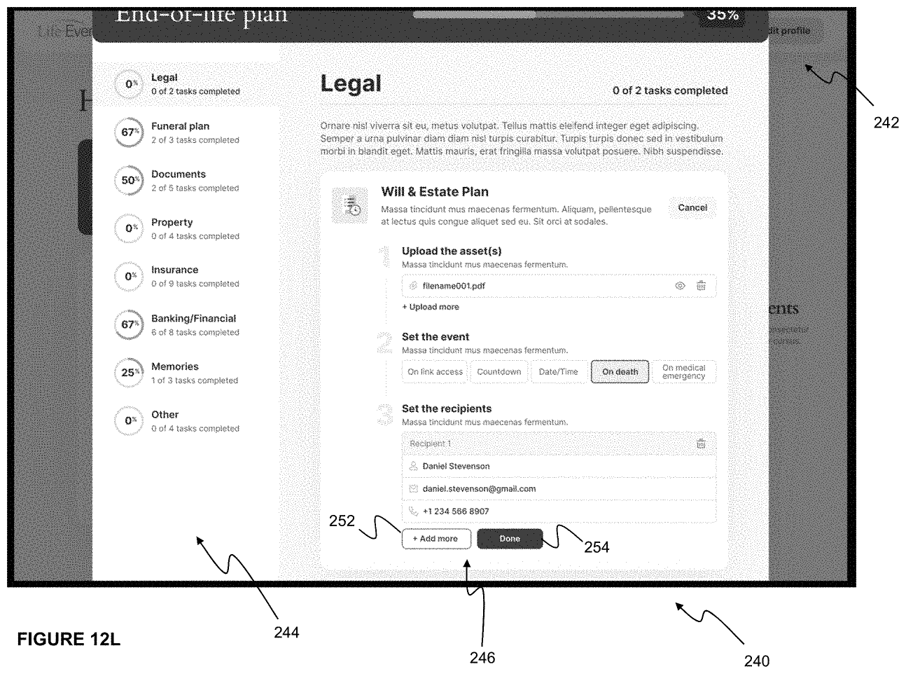

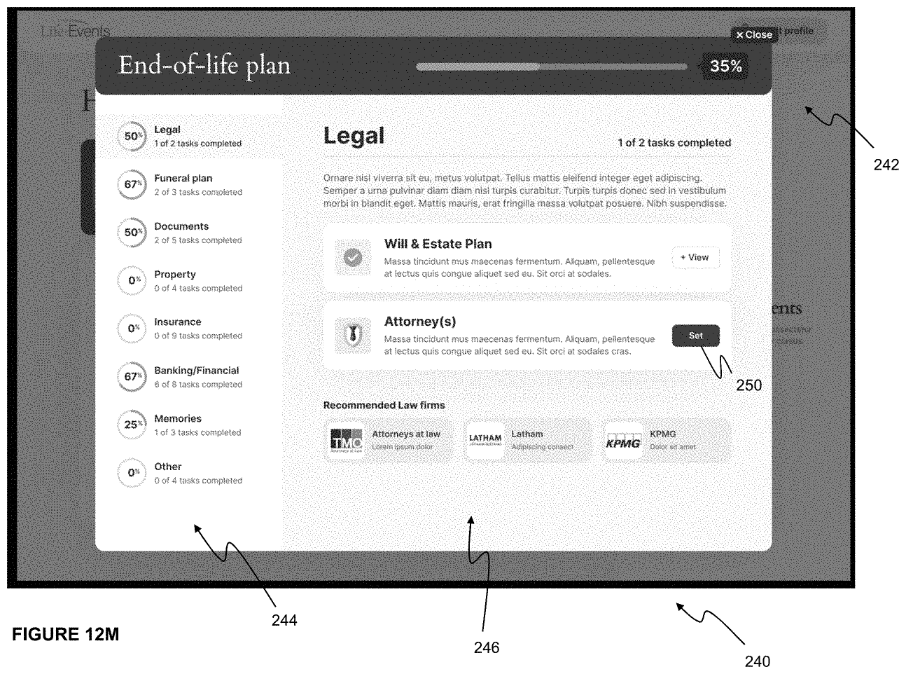

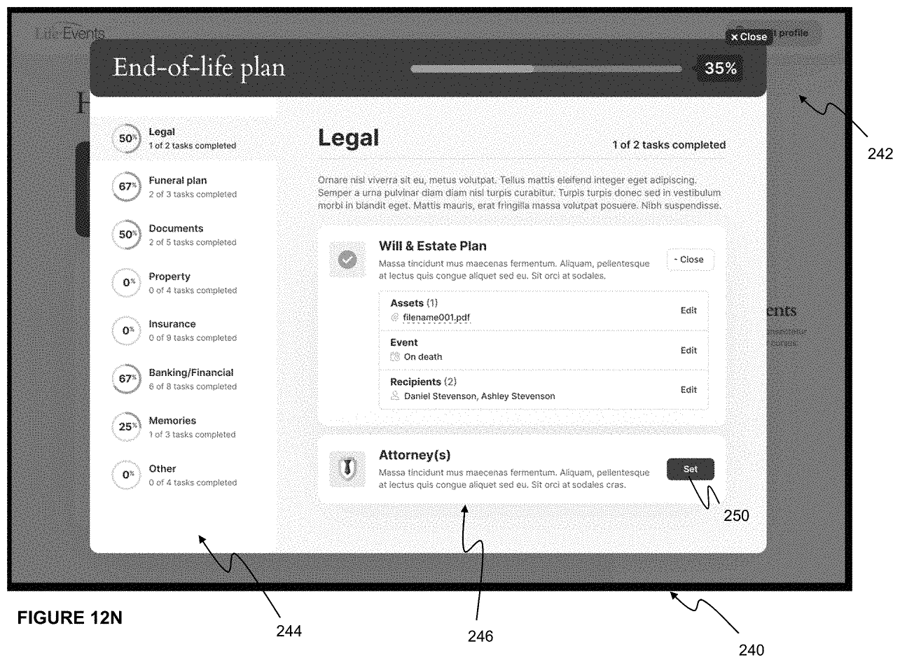

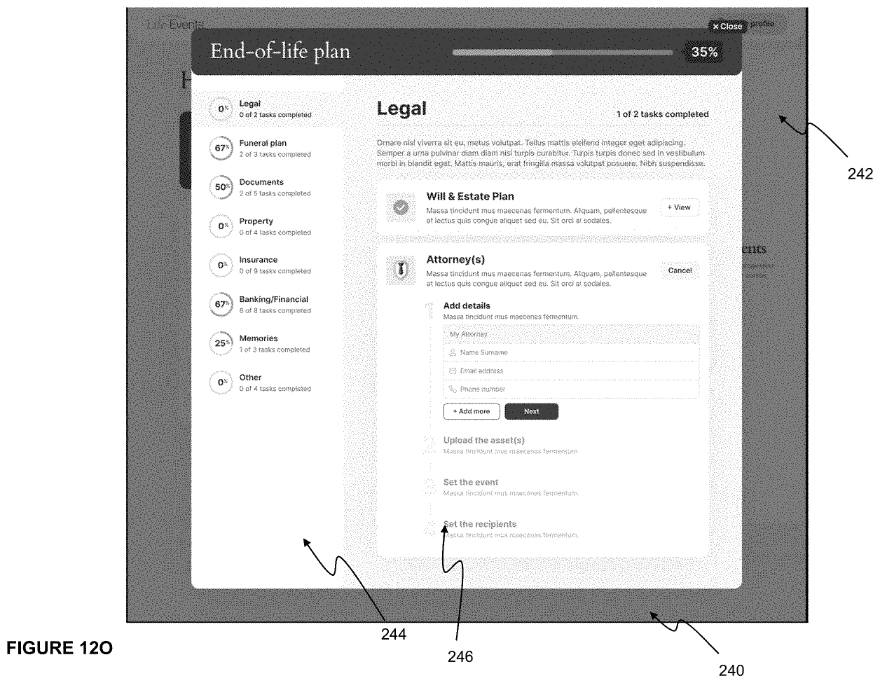

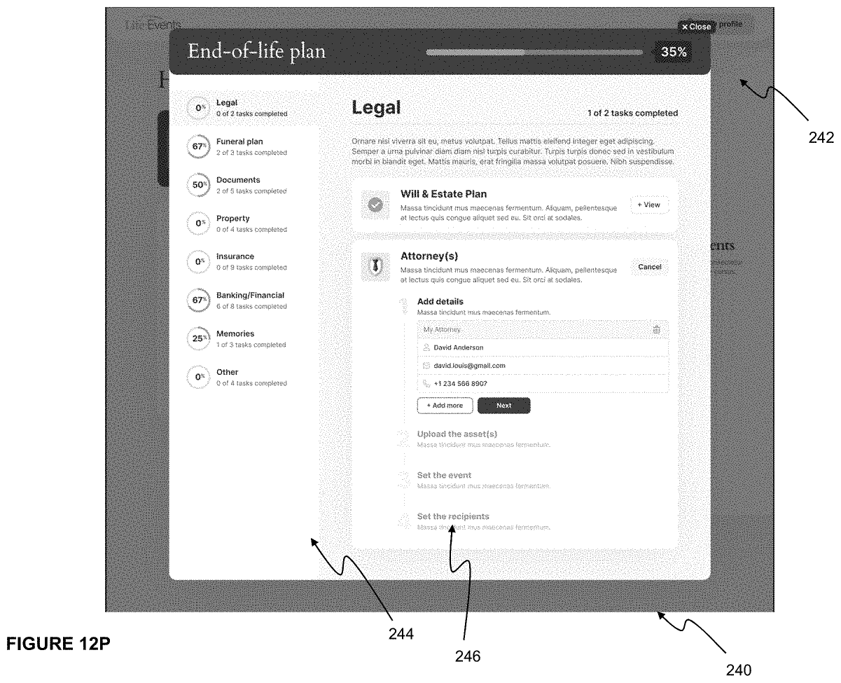

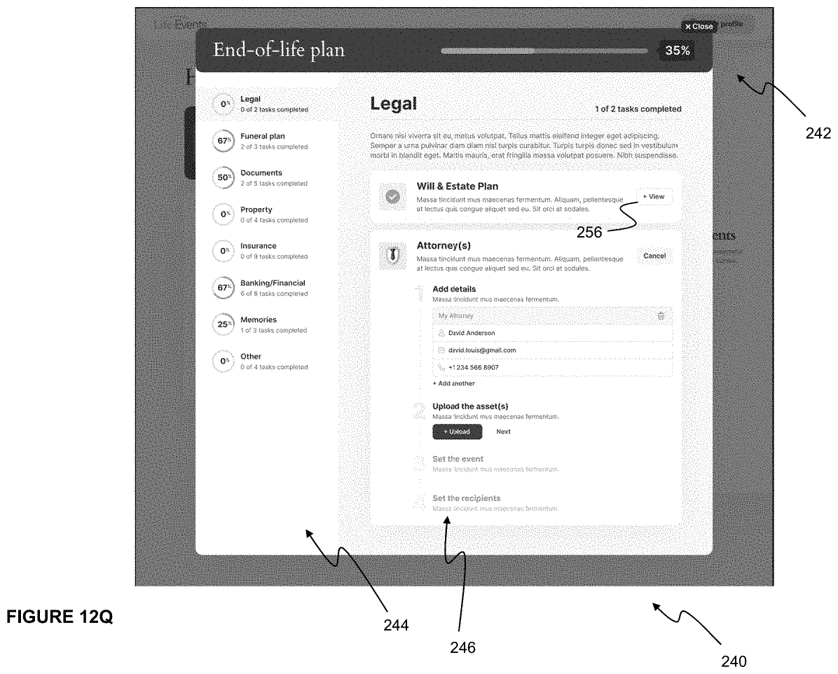

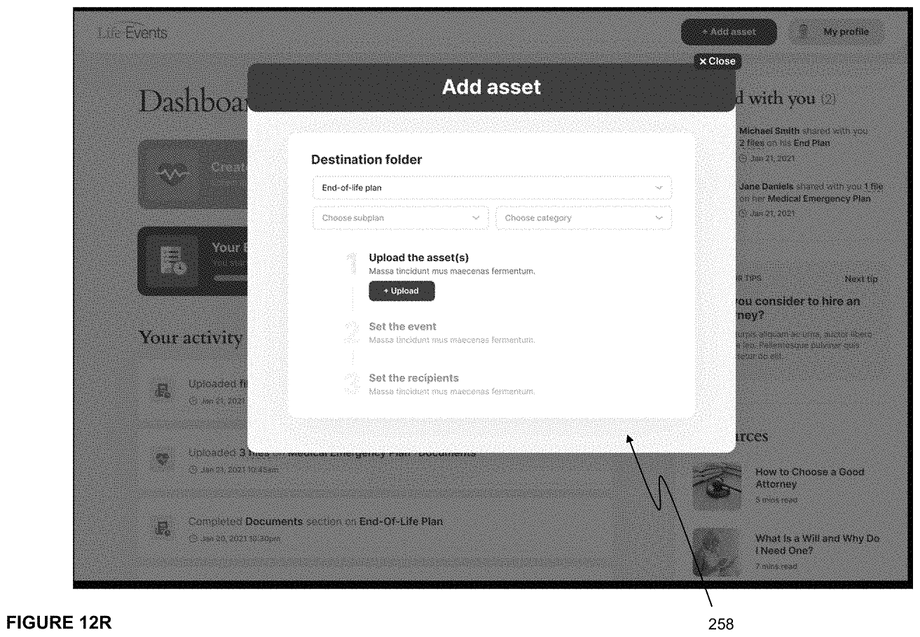

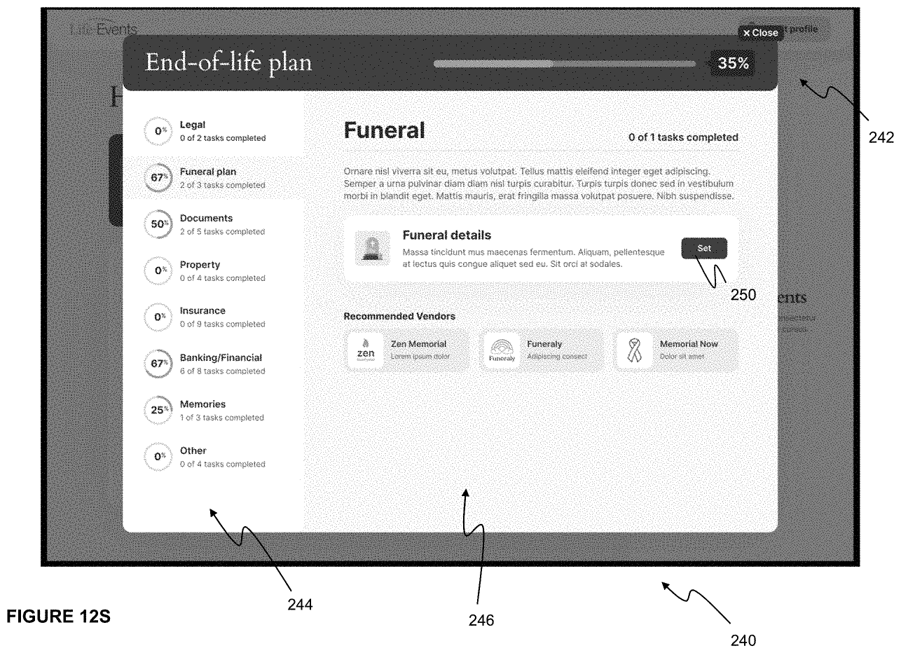

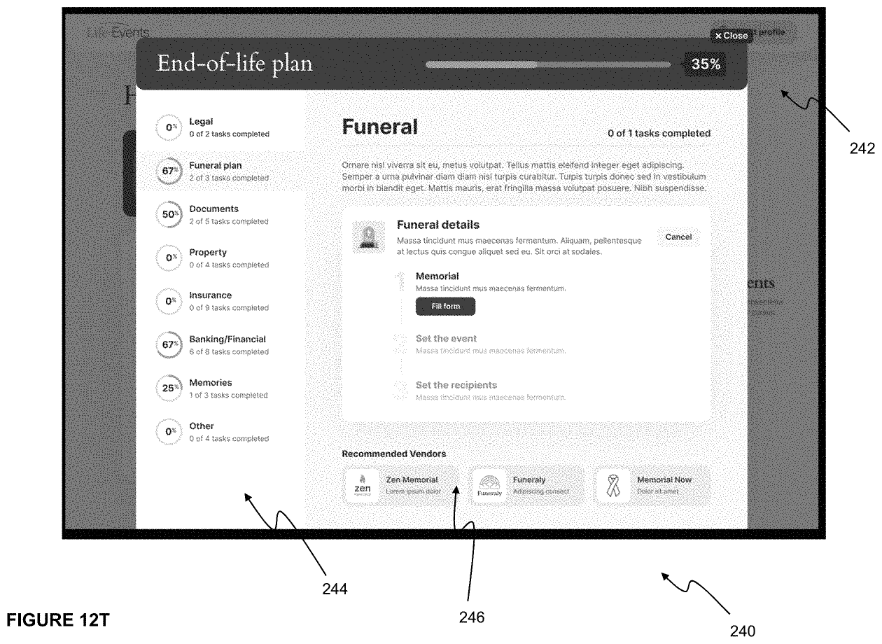

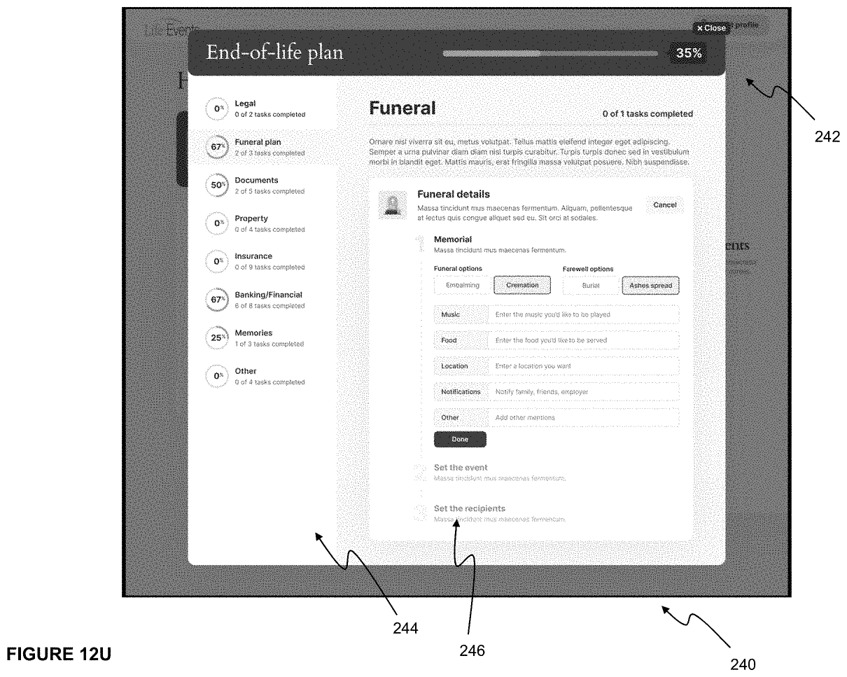

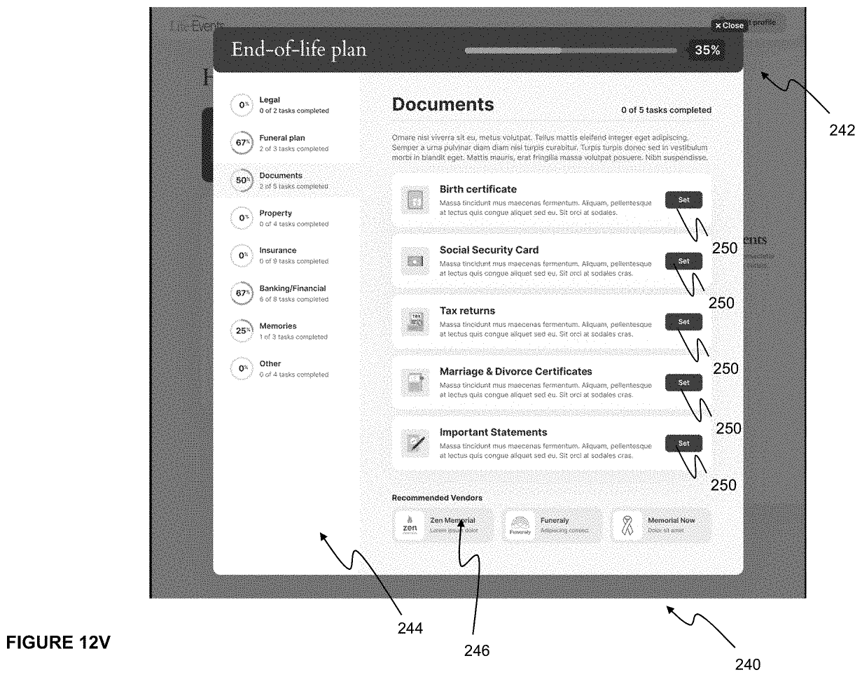

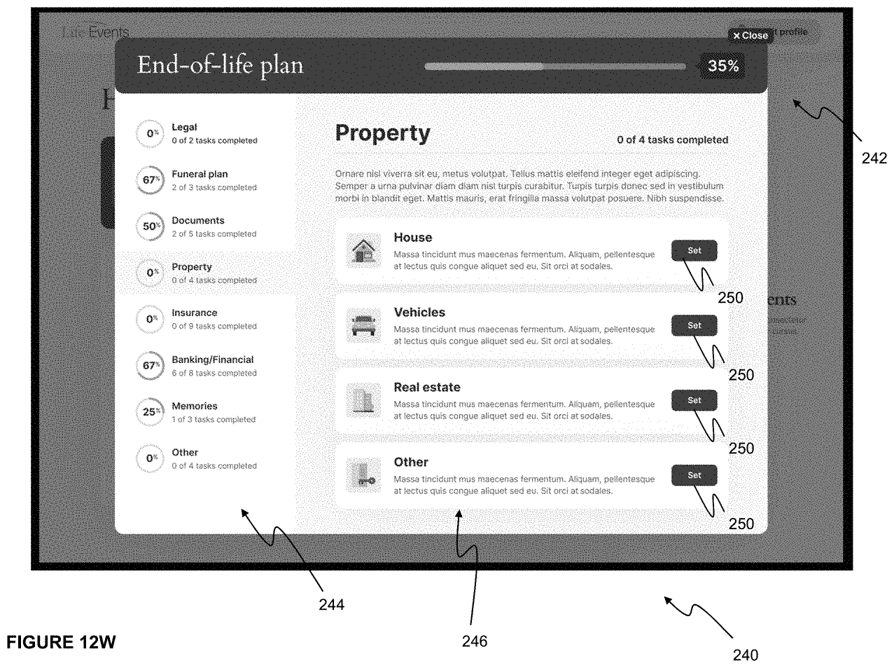

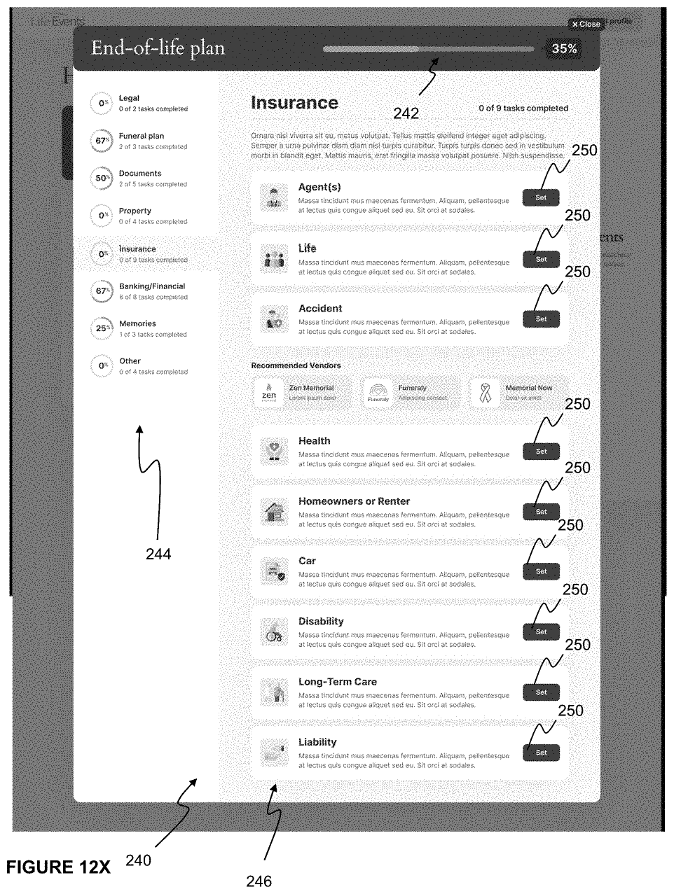

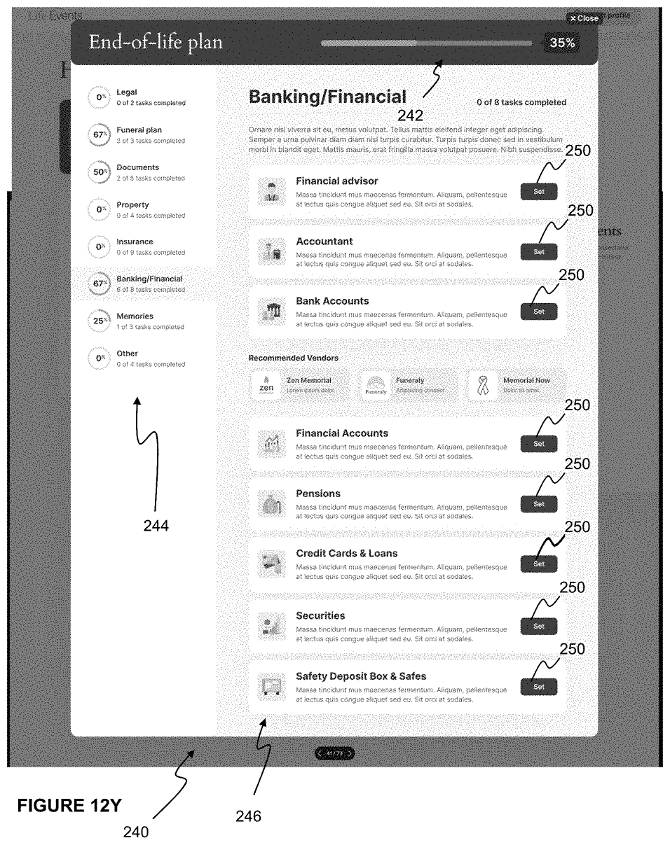

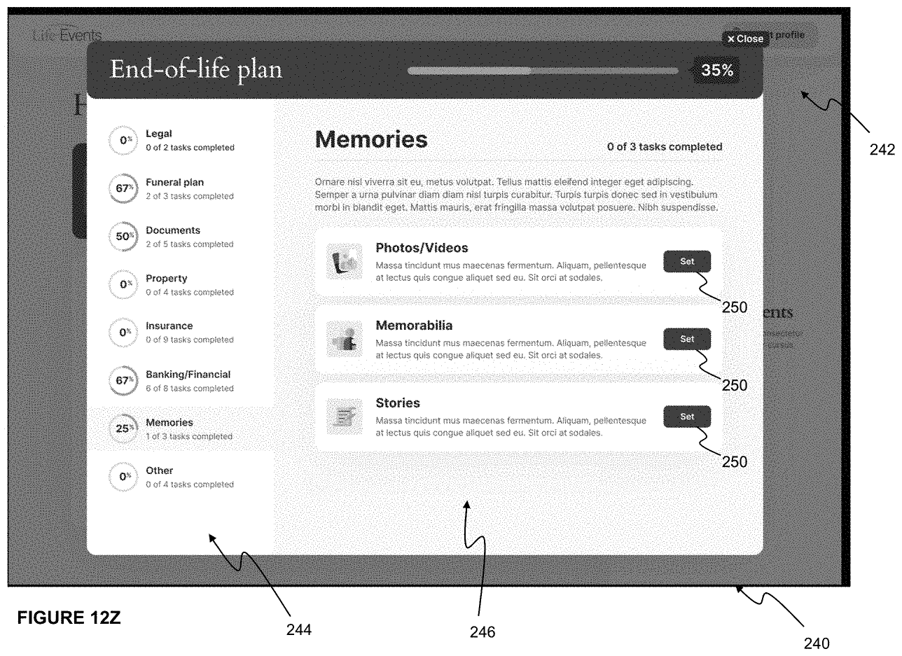

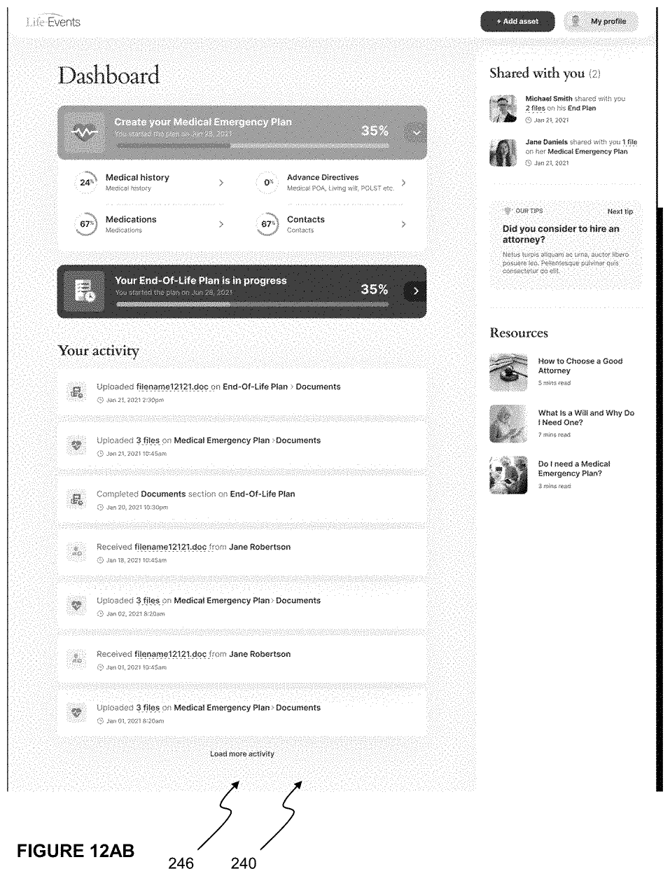

With reference to FIGS. 12A-12AB, an exemplary end-of-life worksheet 240 is illustrated. The illustrated worksheet 240 includes a completion bar 242, a category list 242 and a working section 244.

After the user initiates the end-of-life worksheet 240 (for the first time), the user end-of-life plan is initially. Thus, as shown in FIG. 12A, the completion bar 242 is at 0%. The category list 244 may include a representation of the completion percentage for each category. Initially, each category is also at 0%. The working section 246 presents to the user information pertaining to the next step and may also include other options, e.g., as shown in FIG. 12A, an option to upgrade the user's plan, for example, Tier One to Tier Two. Upon initiation of the end-of-life worksheet 240, the working section includes a Get Started button 248.

As discussed above, each category includes a list of items or digital assets. With reference to FIG. 12B, if the user selects the Get Started button 248, the worksheet 240 will proceed through each category, in turn, in the category list 244 and list the items in the working section 246 that are associated with the current category. Alternatively, the user may click or select on a category in the list category list 244 and the items associated with the selected category will be displayed in the working section 246.

For example, as shown in FIG. 12B, if category “Legal” is the current category, the items associated with the Legal category are listed in the working section 246. Each item has an associated set button 250. Selection of a set button 250 will display the options for the associated item. The options may be dependent upon the user's status or current tier level.

For instance, as shown in FIGS. 12C and 12D, in one embodiment, if the user does not have a subscription all (or most) digital asset files are located in an external repository, e.g., Dropbox. Thus, the user must enter the link to the external repository at which the digital asset is located. Alternatively, if the user has a paid account or subscription, the user may upload the digital files for the associated item in the current category to the internal repository (see FIGS. 12E and 12F).

Once the digital asset has been uploaded or the link saved, the user may set the event trigger (see FIGS. 12G-12J). As discussed above, the event trigger associated with digital assets added via the end-of-life plan worksheet 240 is initially set to “on death”. However, the user may override the default be selecting one of the other event triggers. If the user selects the countdown event trigger option, the user is prompted to enter the countdown period in years, months and/or days (see FIG. 12I). If the user selects the date/time event trigger, the user is prompted to enter a specific date (see FIG. 12J). As shown, an additional event is defined as “on link access”. The system 100 may generate a unique address for a given digital asset. If the address is accessed, the recipient may be given access to the digital asset.

Once the event trigger has been set, the user may set the recipient or recipient(s) for the digital asset. In the illustrated embodiment, the user is prompted to enter the recipient(s)′ details, e.g., name, email address and phone number (see FIGS. 12K-12L). Once the user has entered all details or data for a particular digital asset, the user may enter additional assets under the current category by selecting an add more button 252. Or the user may return proceed to the category list by selecting a done button 254.

After the user selects the done button 254, the items under current category may be displayed in the working section 246 (see FIG. 12M). The user may proceed to the next item (e.g., attorneys), by selected the associated set button 250. If the user selects the set button 250 for the next item, e.g., attorneys, the user is guided through a series of prompts to add information for that item which may include contact information, digital assets, event triggers and recipients (see FIGS. 12O-12Q).

In one aspect of the invention, a user can add digital assets to the end-of-life plan outside of the end-of-life worksheet 240 using an add asset dialog 258 that allows the user to select the plan, e.g., end-of-life plan or medical emergency plan, subplan and category (see FIG. 12R).

To add, view or edit digital assets to a item, the user may select a view button 256 to return to that item (see FIGS. 12M and 12N).

The end-of-life worksheet 240 allows the user to cycle through the rest of the categories and items in turn, as shown:

| Category | FIGS. |
|---|---|
| Funeral | FIGS. 12S-12U |
| Documents | FIGS. 12V |
| Property | FIG. 12W |
| Insurance | FIG. 12X |
| Banking/Financial | FIG. 12Y |
| Memories | FIG. 12Z |

With reference to FIG. 12AB, the home page (or dashboard) 220, may show the current completion level for each plan as well as a list the user's recent activity in the recent activity section 228 and a list of items or digital assets shared with the user (as the recipient) by other users in the shared activity section 230.

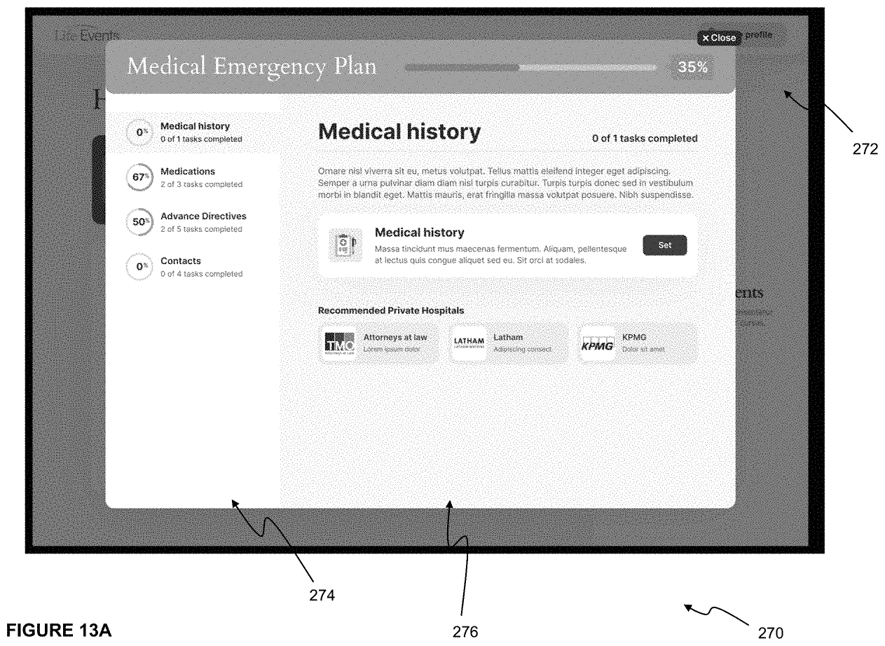

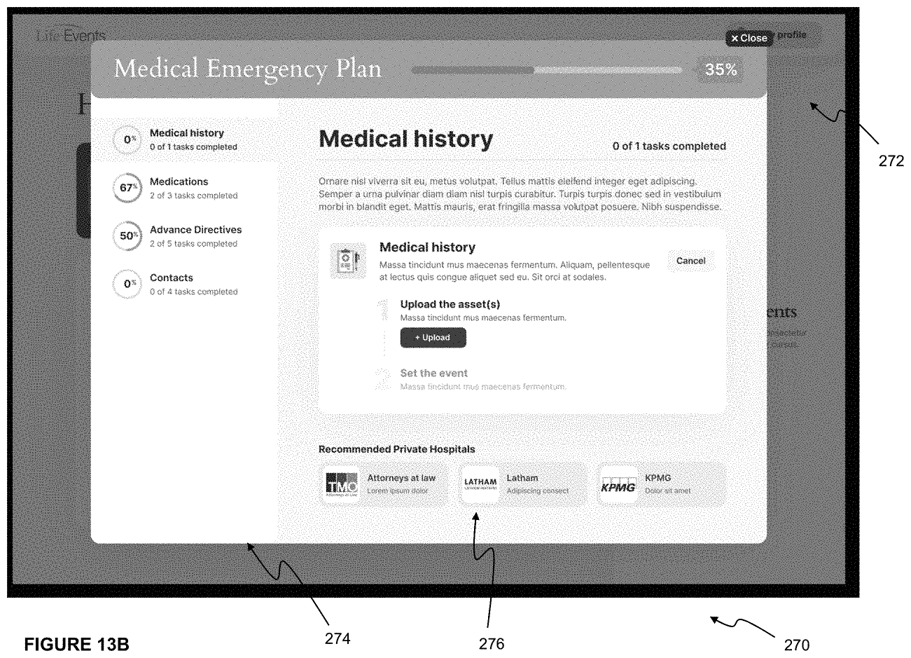

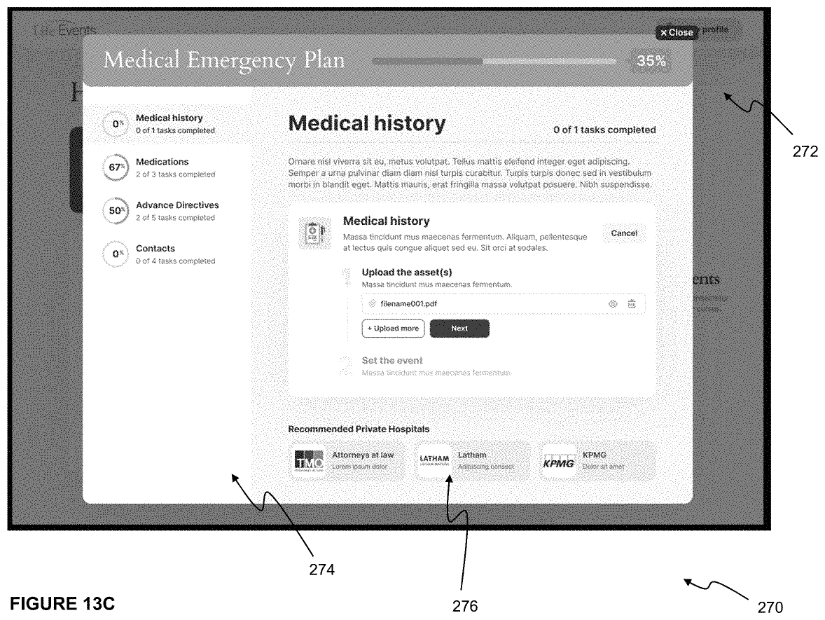

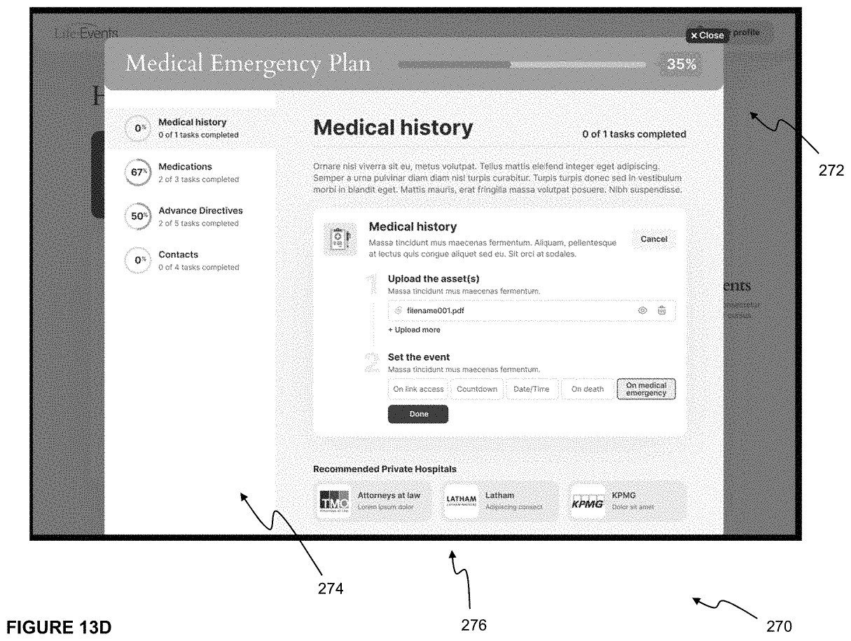

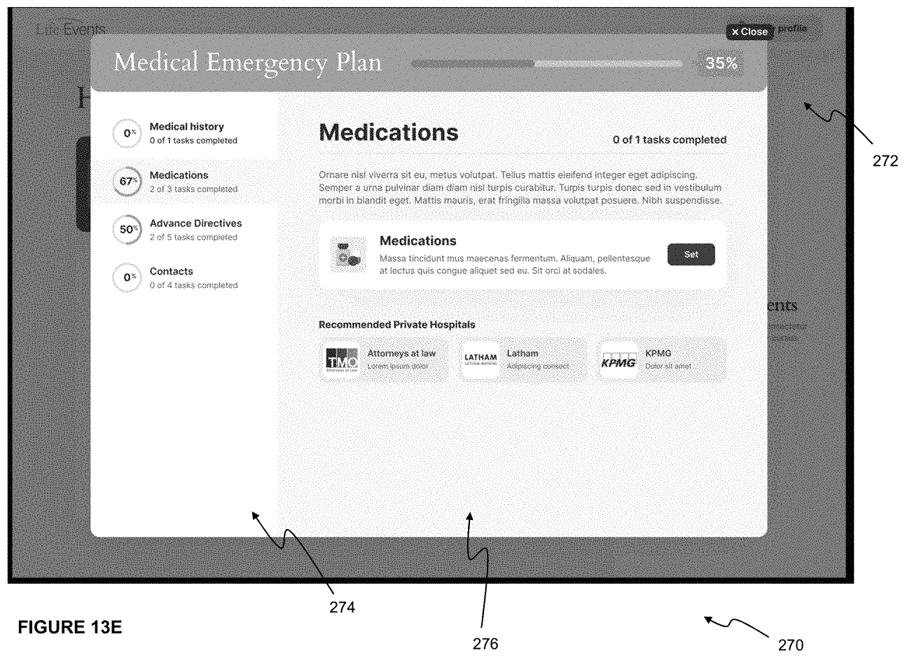

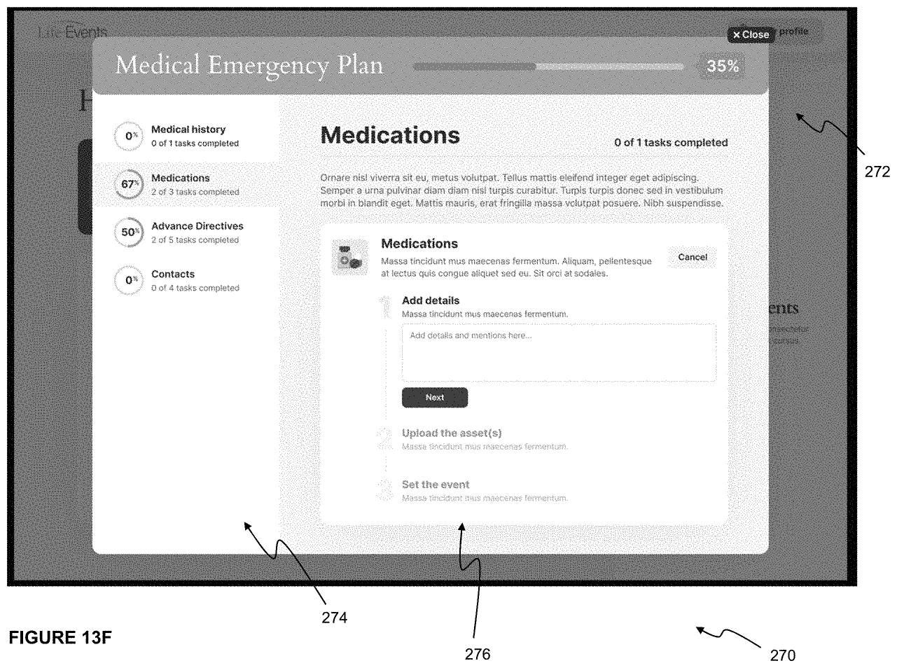

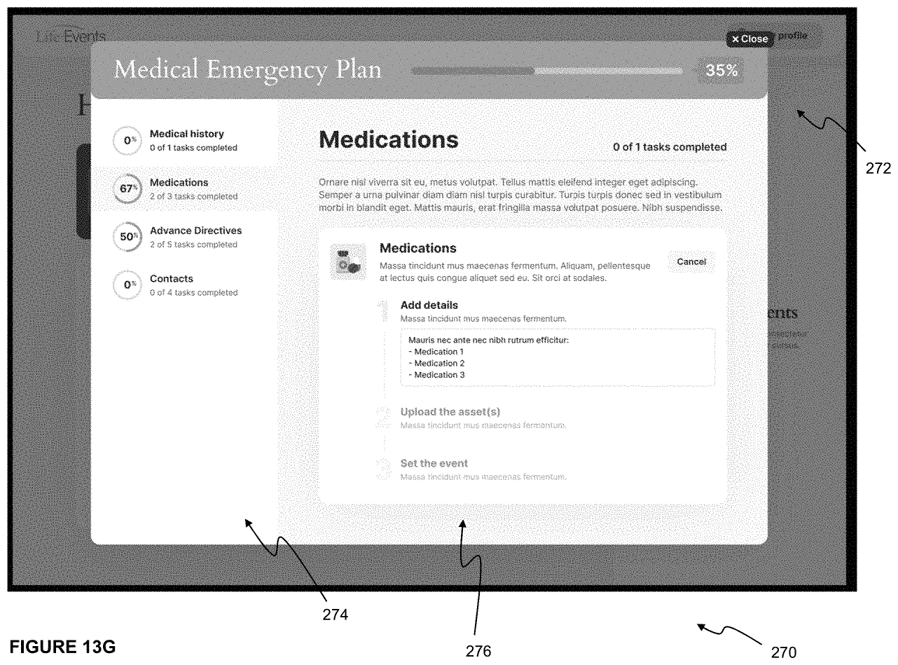

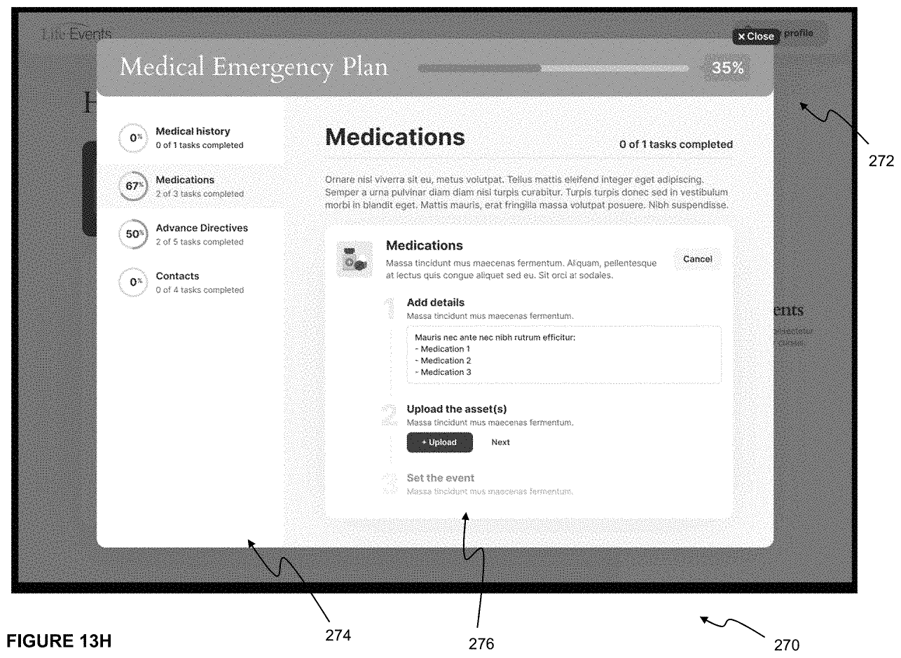

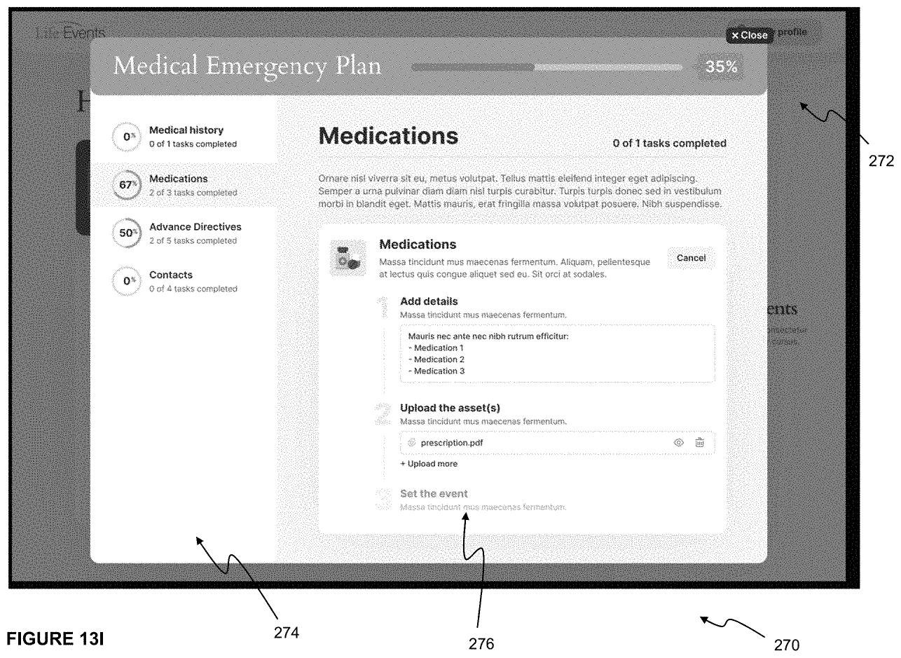

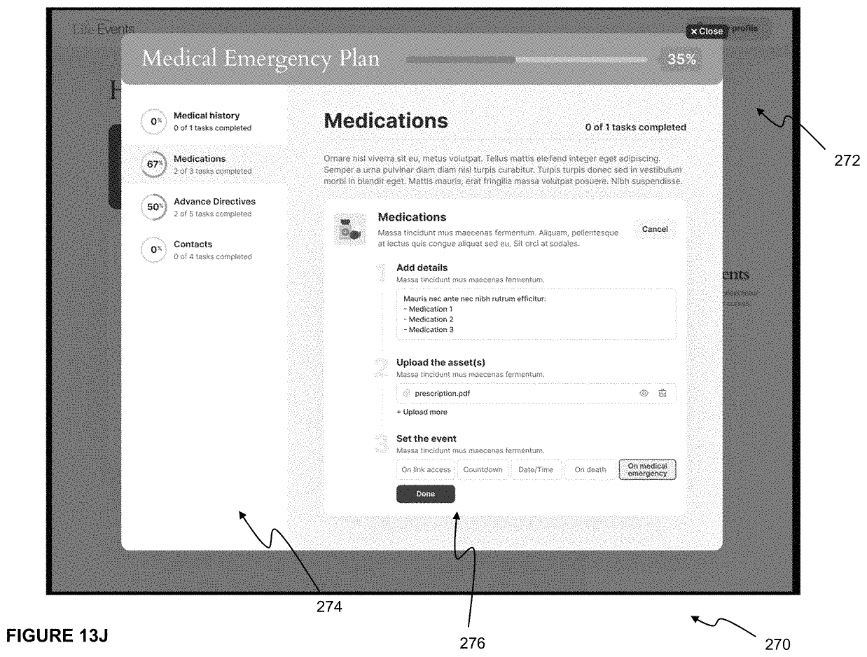

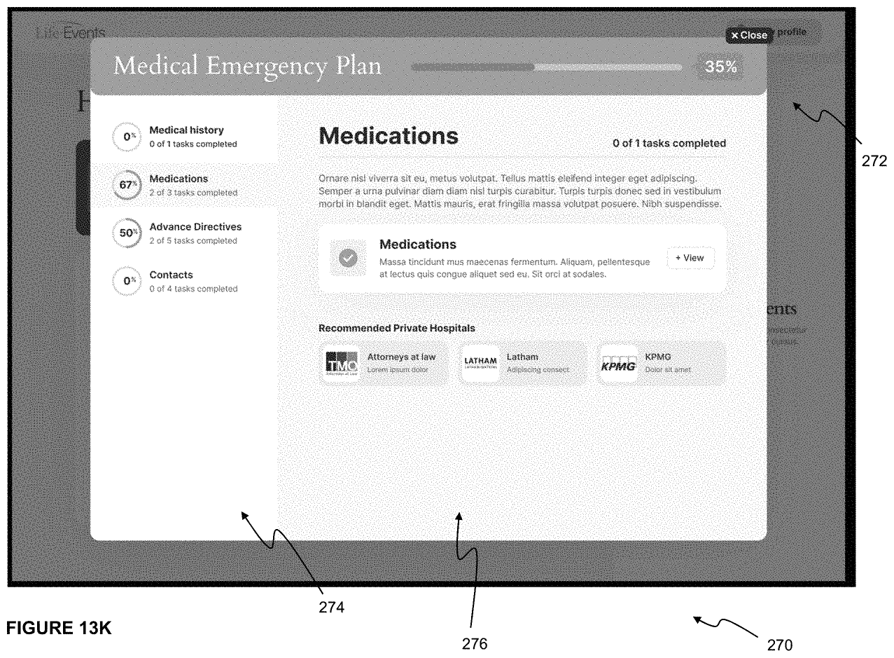

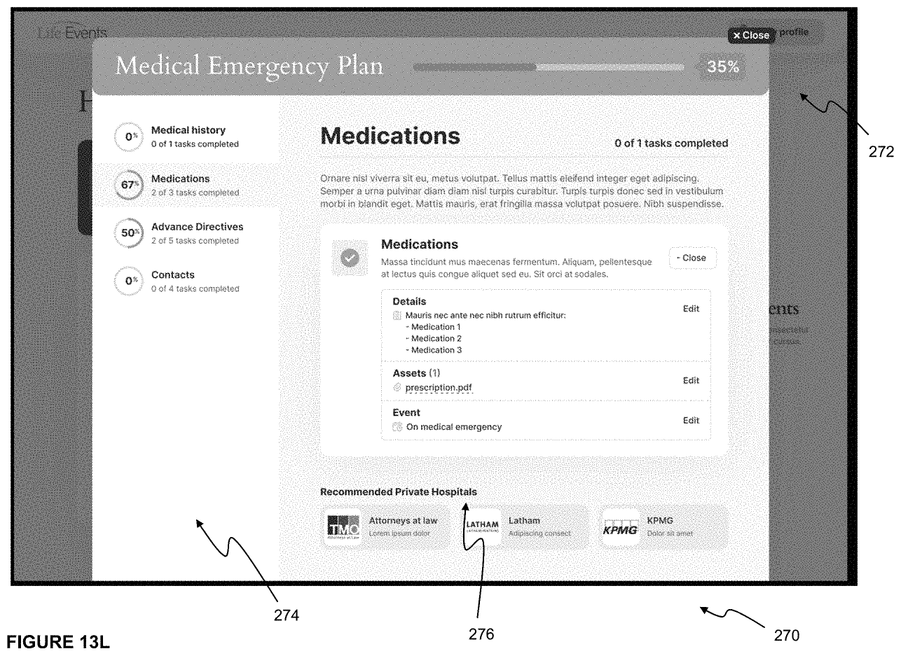

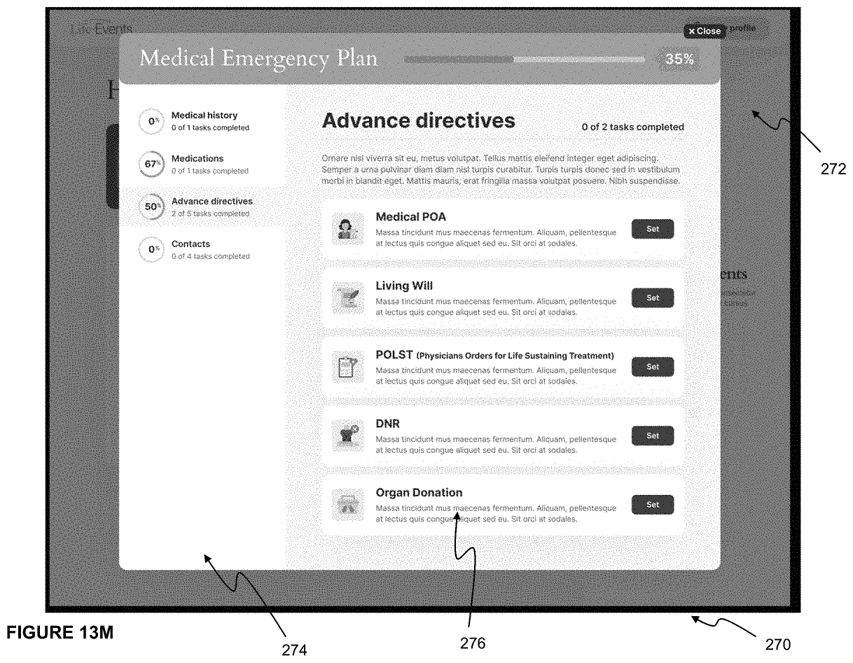

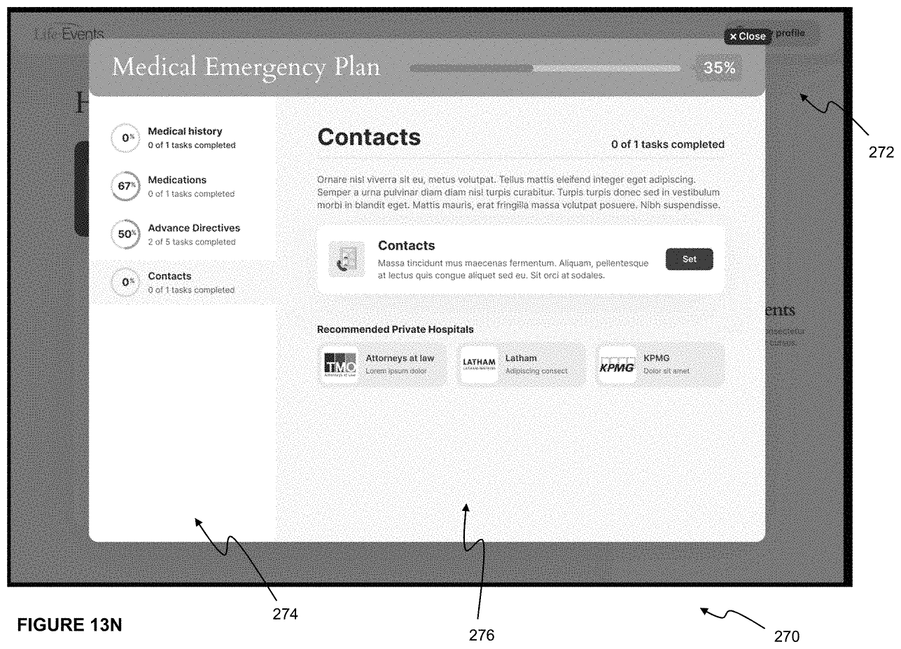

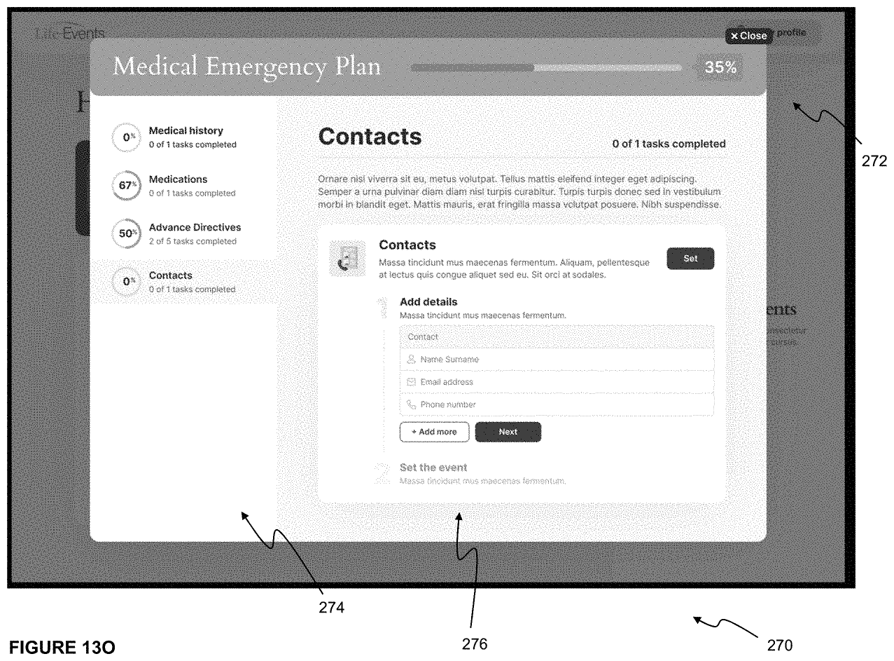

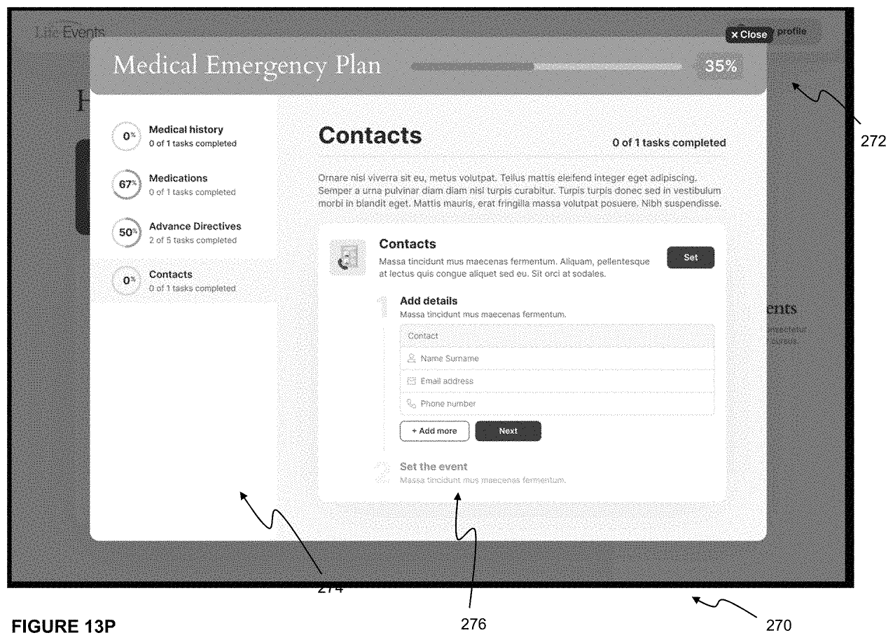

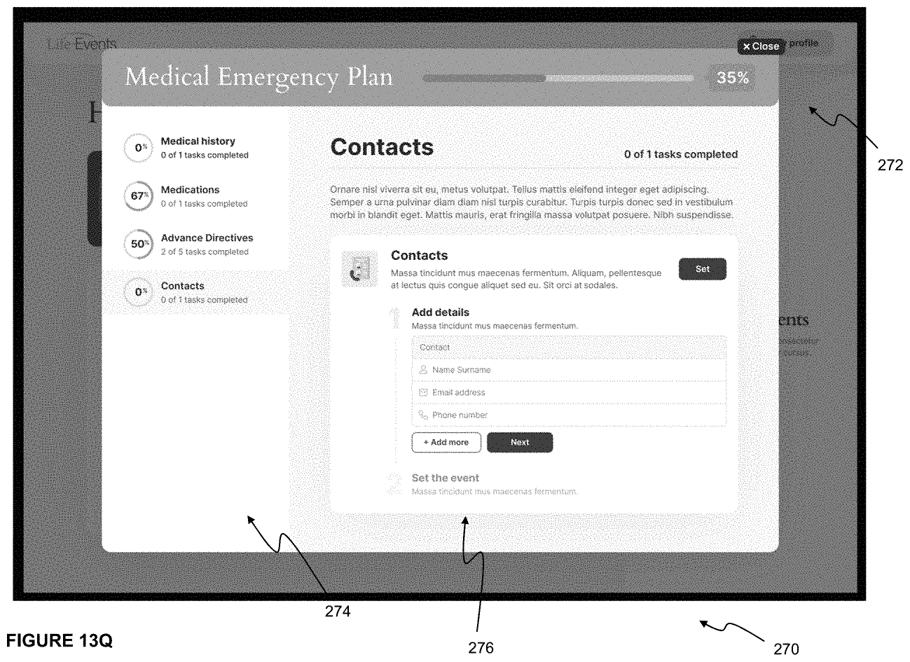

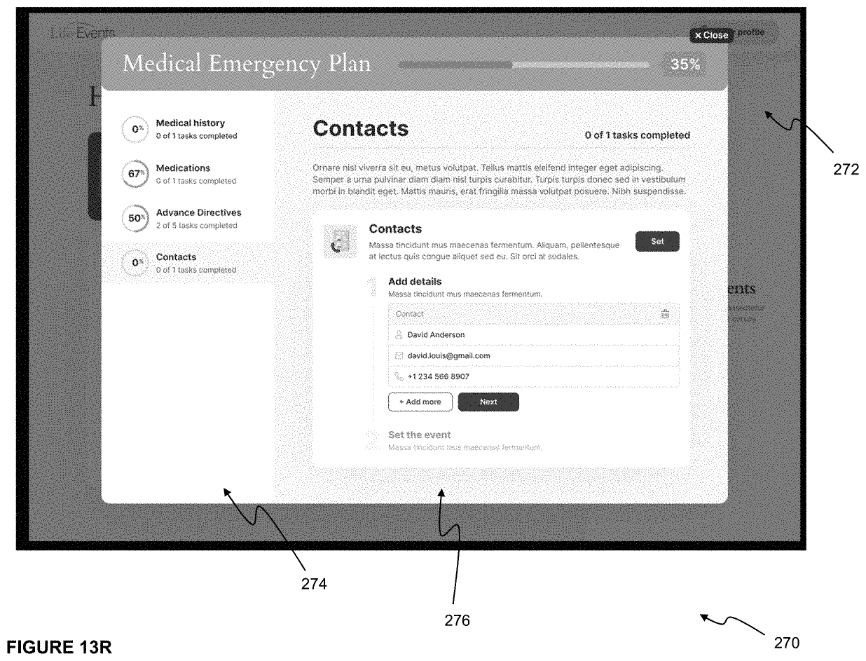

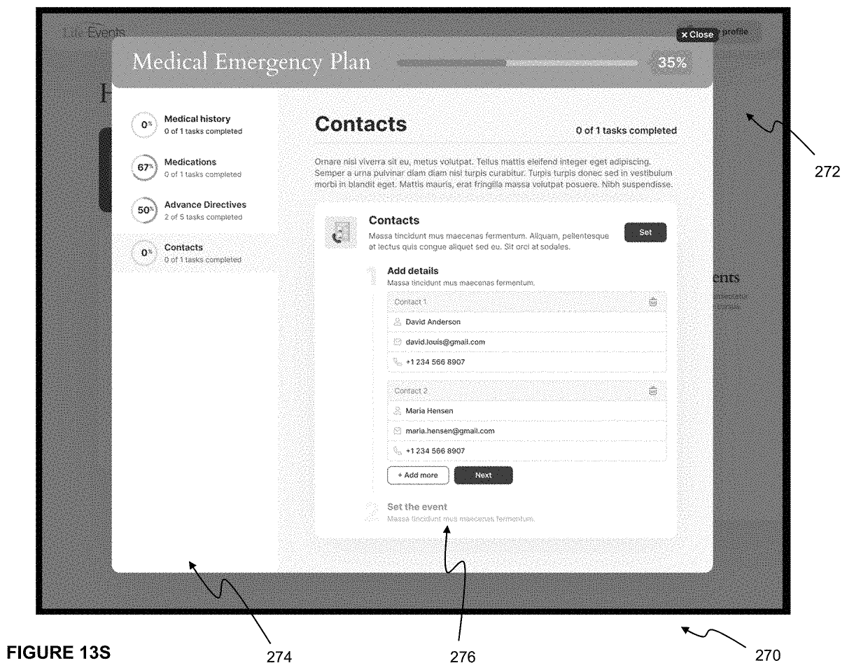

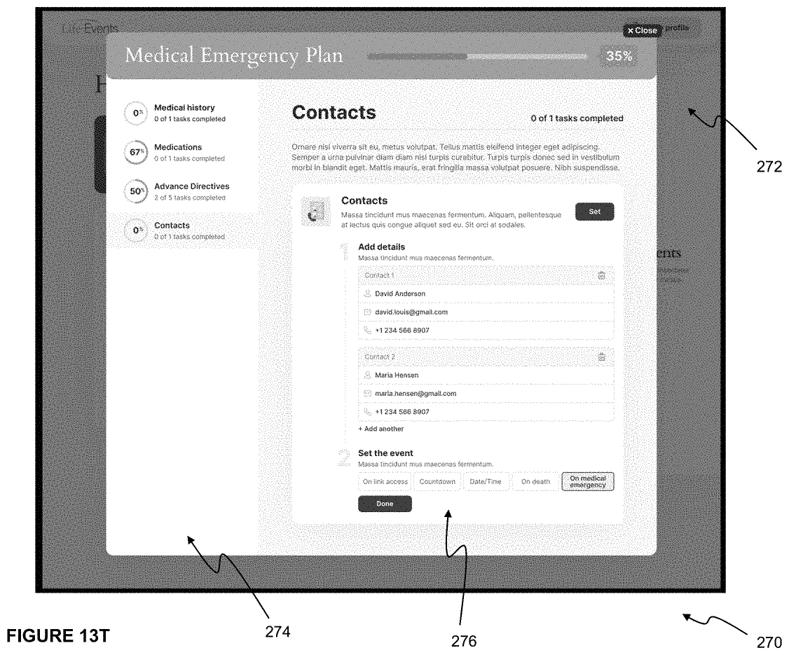

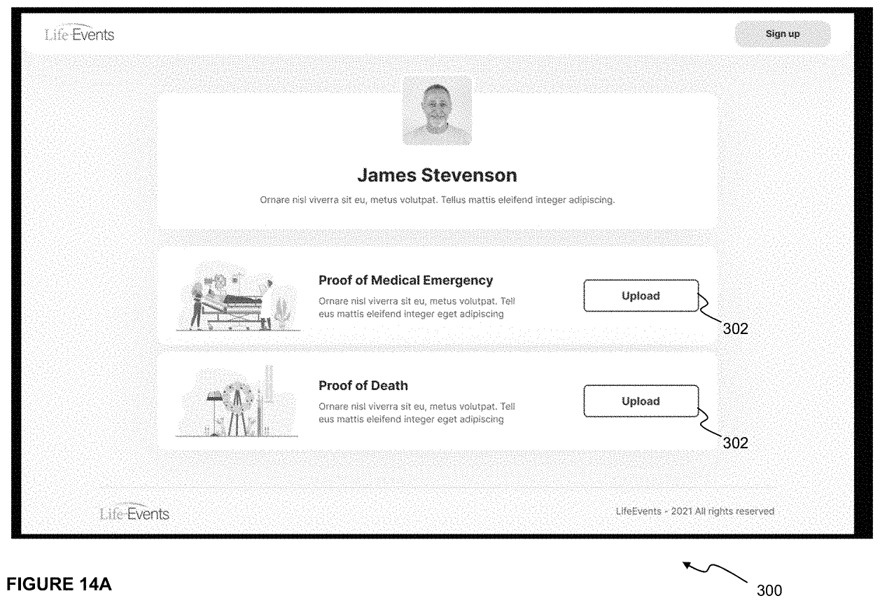

With reference to FIGS. 13A-13T, an exemplary medical emergency worksheet 270 is illustrated. The illustrated medical emergency worksheet 270 includes a completion bar 272, a category list 272 and a working section 274.

After the user initiates the medical emergency worksheet 270 (for the first time), the user medical emergency plan is initially empty, i.e., the completion bar 272 is at 0%. However, as the user proceeds through the worksheet 270 and adds digital assets, the completion bar 272 is modified to reflect the current completion status. As shown in FIG. 13A, for example, the current completion is 35%. As discussed above, the category list 272 includes a list of the predefined sections or categories. Each section or category has a set of defined items or digital assets.

The category list 274 may include a representation of the completion percentage for each category. Initially, each category is also at 0%. The working section 276 presents to the user information pertaining to the next step and may also include other options, e.g., as shown in FIG. 13A links to recommended vendors for particular services.

When the user initiates the medical emergency worksheet 270, the worksheet 270 will proceed through each category, in turn, in the category list 274 and list the items in the working section 276 that are associated with the current category. Alternatively, the user may click or select on a category in the list category list 274 and the items associated with the selected category will be displayed in the working section 276.

For example, as shown in FIG. 13A, the first category in the medical emergency worksheet 270 is “Medical history”. The current category is displayed in the working section 276. To start working on the current category, the user selects a set button 280. Selection of a set button 280 for a particular category will display the options for the associated item. The options may be dependent upon the user's status or current tier level.

In the illustrated embodiment, the medical history category allows the user upload or enter one or more digital assets pertaining to the user's medical history. The process of adding digital assets to under the medical history may be dependent upon the user's current status or tier level. For instance, if the user does not have a subscription all (or most) digital asset files are located in an external repository, e.g., Dropbox. Thus, the user must enter the link to the external repository at which the digital asset is located. Alternatively, if the user has a paid account or subscription, the user may upload the digital files for the associated item in the current category to the internal repository (see FIGS. 13B and 13F).

Once the digital asset has been uploaded or the link saved, the user may set the event trigger (see FIG. 13D). As discussed above, the event trigger associated with digital assets added via the medical emergency worksheet 270 is initially set to “on medical emergency”. However, the user may override the default be selecting one of the other event triggers. If the user selects the countdown event trigger option, the user is prompted to enter the countdown period in years, months and/or days. If the user selects the date/time event trigger, the user is prompted to enter a specific date. As shown, an additional event is defined as “on link access”. The system 100 may generate a unique address for a given digital asset. If the address is accessed, the recipient may be given access to the digital asset.

A recipient is not needed for the medical emergency event trigger or the on link access. However, one of the other event triggers are specified, the user may set the recipient or recipient(s) for the digital asset (see above). In the illustrated embodiment, the user is prompted to enter the recipient(s)′ details, e.g., name, email address and phone number (see FIGS. 12K-12L). Once the user has entered all details or data for a particular digital asset, the user may enter additional assets under the current category by selecting an add more button 252. Or the user may return proceed to the category list by selecting a done button 254. Using an upload more button 282, the user may add additional digital assets or files that utilize the same event trigger as a medical history. To move to the next category, the user may use a next button 284.

In one aspect of the invention, a user can add digital assets to the end-of-life plan outside of the end-of-life worksheet 270 using an add asset dialog 258 (see above).

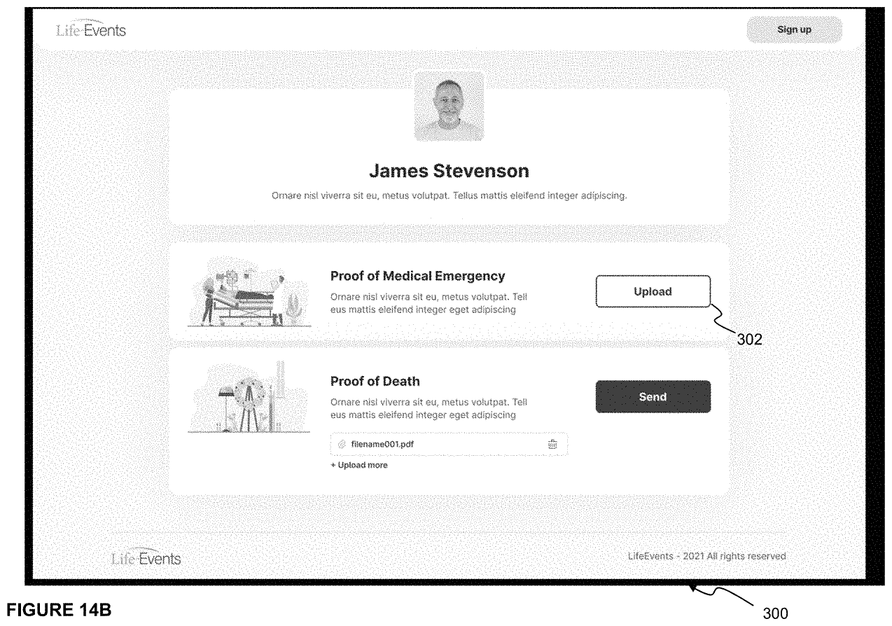

The medical event worksheet 270 allows the user to cycle through the rest of the categories and items in turn, as shown:

| Category | FIGS. |
|---|---|
| Medication | FIGS. 13E-13L |
| Advance Directives | FIGS. 13M |
| Contacts | FIG. 13N-14T |

With reference to FIGS. 14A-14B, an exemplary user public profile 300 is shown. The public profile 300 may be accessed via the public website 200 or via a link provided by the user. In the event of either a medical emergency (of the user) or the death of the user, any person via the user public profile 300 may upload to the system 100 a death certificate or proof of a medical emergency via respective upload buttons 302. With specific reference FIG. 8C, a method M600 for initiating a user end-of-life plan or a medical emergency plan is shown. In a first step S602, a person may access a user public profile through the system 100. On the user public profile, the person may elect to initiate either an end-of-life event or a medical emergency event associated with the user in a second step S604. In a third step S606, the person may upload either a death certificate or proof of a medical emergency. In one aspect of the present invention, the death certificate or proof of medical emergency must be verified manually. In another aspect of the present invention, the death certificate or proof of medical emergency may be automatically verified, e.g., by comparison or look up of the Death Certificate in an external database or the person who requested access in a registry of licensed medical professionals, respectively. Once verified, a confirmation message is sent in a fourth step S608.

The user public profile are all public, i.e., anyone with the link can access it. If there are public documents that have been added to a user's profile, they will be visible here to anyone with the profile link. But if there are assets that only became available on a specific Event, like death, the viewer of this profile page will have to login with their username and password (the email provided must match the email the initial user entered when adding that recipient) in order to see those materials. Still, they will only be able to see digital assets for which they were added as a recipient.

With specific reference to FIG. 8D, a method M700 associated with notifying a recipient that the user had provided or sent the recipient a digital asset (and that the triggering event has been triggered) is shown. In a first step S702, a communication is sent to the recipient, typically, an email or text message. In a second step S704, the recipient may logon to the system 100 or create an account, and then logon. In a third step S706, the system 100 provides access to the digital asset.

With reference to FIG. 8E, a method M800 associated with an administrator is shown. The administrator may access or logon to the system 100 in a first step S802. In a second step S804, an administrator dashboard is displayed. Using the administrator dashboard, the administrator may perform the following functions:

S806—Manage user accounts (e.g., assist with lost passwords, delete accounts, etc. . . . );

S808—Review and approve death certificates and/or medical emergency documents;

S810—Create, manage, edit worksheets; and

S812—Access user analytics.

Reference throughout this specification to “one embodiment”, “an embodiment”, “one example” or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

Embodiments in accordance with the present invention may be embodied as an apparatus, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “module” or “system.” Furthermore, the present invention may take the form of a computer program product embodied in any tangible media of expression having computer-usable program code embodied in the media. An apparatus may be expressed in terms of modules and/or units that include one or more discrete hardware components or portions thereof as configured by software (in any form). Furthermore, an apparatus may take the form of one or more elements expressed as a means for performing a specified function. When expressed in such a form, the means are to be interpreted as meaning the combination of hardware components or portions thereof contained within this specification, and any equivalents thereof.

Any combination of one or more computer-usable or computer-readable media (or medium) may be utilized. For example, a computer-readable media may include one or more of a portable computer diskette, a hard disk, a random access memory (RAM) device, a read-only memory (ROM) device, an erasable programmable read-only memory (EPROM or Flash memory) device, a portable compact disc read-only memory (CD-ROM), an optical storage device, and a magnetic storage device. Computer program code for carrying out operations of the present invention may be written in any combination of one or more programming languages.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

The flowchart and block diagrams in the flow diagrams illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions. These computer program instructions may also be stored in a computer-readable media that can direct a computer or other programmable data processing apparatus to function in a particular manner, such that the instructions stored in the computer-readable media produce an article of manufacture including instruction means which implement the function/act specified in the flowchart and/or block diagram block or blocks.

Several (or different) elements discussed below, and/or claimed, are described as being “coupled”, “in communication with”, or “configured to be in communication with”. This terminology is intended to be non-limiting, and where appropriate, be interpreted to include without limitation, wired and wireless communication using any one or a plurality of a suitable protocols, as well as communication methods that are constantly maintained, are made on a periodic basis, and/or made or initiated on an as needed basis. The term “coupled” means any suitable communications link, including but not limited to the Internet, a LAN, a cellular network, or any suitable communications link. The communications link may include one or more of a wired and wireless connection and may be always connected, connected on a periodic basis, and/or connected on an as needed basis.

A controller, computing device, server or computer, such as described herein, includes at least one or more processors or processing units and a system memory (see above). The controller typically also includes at least some form of computer readable media. By way of example and not limitation, computer readable media may include computer storage media and communication media. Computer storage media may include volatile and nonvolatile, removable and non-removable media implemented in any method or technology that enables storage of information, such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art should be familiar with the modulated data signal, which has one or more of its characteristics set or changed in such a manner as to encode information in the signal. Combinations of any of the above are also included within the scope of computer readable media.

The order of execution or performance of the operations in the embodiments of the invention illustrated and described herein is not essential, unless otherwise specified. That is, the operations described herein may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In some embodiments, a processor, as described herein, includes any programmable system including systems and microcontrollers, reduced instruction set circuits (RISC), application specific integrated circuits (ASIC), programmable logic circuits (PLC), and any other circuit or processor capable of executing the functions described herein. The above examples are exemplary only, and thus are not intended to limit in any way the definition and/or meaning of the term processor.

In some embodiments, a database, as described herein, includes any collection of data including hierarchical databases, relational databases, flat file databases, object-relational databases, object oriented databases, and any other structured collection of records or data that is stored in a computer system. The above examples are exemplary only, and thus are not intended to limit in any way the definition and/or meaning of the term database. Examples of databases include, but are not limited to only including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Wash.; and Sybase is a registered trademark of Sybase, Dublin, Calif.).

The above description of illustrated examples of the present invention, including what is described in the Abstract, are not intended to be exhaustive or to be limitation to the precise forms disclosed. While specific embodiments of, and examples for, the invention are described herein for illustrative purposes, various equivalent modifications are possible without departing from the broader spirit and scope of the present invention.

It is to be appreciated that the terms “include,” “includes,” and “including” have the same meaning as the terms “comprise,” “comprises,” and “comprising.”

### INDUSTRIAL APPLICABILITY

With reference to the drawings and in operation, a digital asset management system 100 is provided. The purpose of the digital asset management system 100 is to move important files and information from one person to another upon some designated trigger, or “Event.” The primary use-cases would be death—or incapacitation, which may be referred to as a medical emergency, but could encompass other scenarios: for instance, a particular day/time, or the expiration of a countdown timer.

The digital asset management system 100 may allow users to upload important documents and other information, i.e., digital assets, set event (and define the event triggers) upon which those digital assets should be distributed, and designate the recipient(s) that will receive those digital assets once the Event occurs.

After users sign up and create an account, the user will be directed to a home page that consists of all of the events the user has set up, and the events for which other users have added them as a recipient. On the top of the home page, users will see a prompt that says “Do you have an End Plan?” and another that asks “Do you have a Medical Emergency Plan?” From there, they can navigate to a end-of-life worksheet or a medical emergency worksheet page (see above) in order to get started.

On the end-of-life worksheet, the user will see a series of prompts that encourage them to add documents, notes, and instructions (“digital assets”) pertaining to different categories of information that will be important to the user's friends, family, and caregivers after death. These categories may, but are not limited to healthcare, legal, financial, memorial, and legacy considerations—with items spanning people, locations, documents, accounts, and other (these get populated as they are added in the above categories). The medical emergency worksheet will follow a similar format but will focus specifically on medical information. In one aspect of the present invention, paid users can store files directly on the system 100 in an internal repository. For non-paid users, files may be stored through third party solutions like Google Drive or Dropbox. The system 100 will include a link to these file locations.

For each digital asset a user adds, the user will be prompted to set the event and/or event trigger for that digital asset (i.e., the event on which the item will be shared with the intended recipient(s)), and the recipient(s) for that item (i.e., who the item will be shared with once the Event occurs). When setting the Event, the user can choose from a variety of options: they can set a countdown timer or a specific date/time, create a public share link, or specify that the item should only be shared on death or on incapacitation (i.e. ‘Medical Emergency’). Digital assets added through the end-of-life worksheet will default to the “on death” event type, and assets added through the medical emergency worksheet will default to the “on Medical Emergency” Event type.

When an Event occurs, all recipient(s) will receive a notification that tells the recipient how/where to access the digital assets for which they have been added as a recipient. If the Event was set as an end-of-life or medical emergence event, someone must first provide Proof of Death (Death Certificate) or Proof of Medical Emergency and a platform administrator may manually verify that it is authentic. Alternatively, the Proof of Death or Medical Emergency may be automatically verified (see above). In one aspect of the present invention, any person can upload Proof of Death or Proof of Medical Emergency by visiting the user public profile.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A digital asset management system, comprising:
a computer system having a processing device;
data storage media coupled to the computer system for storing a computer program defining an algorithm; and,
a user application coupled to the computer system and being associated with a user device, the user application having a user interface, the processing device being in communication with the user application and including:
an account creation module defined by the computer program and being configured to allow the user to create a user account using the user interface of the user application,
an event creation module defined by the computer program and being configured to allow the user to access a user account and to create an event associated with the user account, the event including an event trigger and digital asset access information associated with a digital asset, and
an event trigger module defined by the computer program and being configured to perform the steps of:
establish an indication that the event trigger has occurred,
responsively provide access to the digital asset to a recipient using the digital asset access information.

2. A digital asset management system, as set forth in claim 1 wherein the event creation module is configured to send a message to the recipient when the event is created.

3. A digital asset management system, as set forth in claim 2, wherein the message includes the user and the nature of the event trigger.

4. A digital asset management system, as set forth in claim 1, wherein the digital asset is a digital reference to a physical item.

5. A digital asset management system, as set forth in claim 4, wherein the digital reference includes a digital message associated with the physical item.

6. A digital asset management system, as set forth in claim 1, wherein the event trigger module establishes an indication that the event trigger has occurred by proactively scanning for the trigger event.

7. A digital asset management system, as set forth in claim 1, wherein the event trigger module establishes an indication that the event trigger has occurred in response to receive a signal from outside of the system.

8. A digital asset management system, as set forth in claim 1, further comprising a database, wherein the event creation module is configured to perform the steps of:
allowing the user to enter an associated username and password and storing the username and password in a user data record associated with the user in the database,
allowing the user to enter asset data associated with the digital asset and storing the asset data in an asset record in the database.

9. A digital asset management system, as set forth in claim 8, further comprising a digital repository and the event creation module is configured to perform the step of allowing the user to upload the digital asset to the digital repository.

10. A digital asset management system, as set forth in claim 8, wherein the digital asset is stored in an external repository and the asset data includes a location and access details related to the digital asset in the external repository.

11. A digital asset management system, as set forth in claim 1, wherein the event trigger is one or more of (a) a countdown timer, (b) a specific date and time, (c) death and (d) incapacitation.

12. A digital asset management system, as set forth in claim 11, wherein the event creation module is further configured to allow the user to establish recipient data for the identified recipient for the digital asset, wherein the recipient data includes contact information.

13. A digital asset management system, as set forth in claim 12, wherein the event creation module is further configured to send a communication to the recipient in response to establishment that the indication of the event trigger has occurred.

14. A digital asset management system, as set forth in claim 13, wherein, if the event trigger is death, proof of death must be received by the event trigger module for the event trigger indication to be established.

15. A digital asset management system, as set forth in claim 14, wherein the proof of death is manually verified.

16. A digital asset management system, as set forth in claim 14, wherein the proof of death is automatically verified against an external database of death records.

17. A digital asset management system, as set forth in claim 11, wherein, if the event trigger is incapacitation, then proof of incapacitation must be received by the event trigger module for the event trigger indication to be established.

18. A digital asset management system, as set forth in claim 17, wherein the proof of incapacitation must be provided by a licensed medical professional.

19. A digital asset management system, as set forth in claim 18, wherein the system manually or automatically confirms that the proof of incapacitation was uploaded by a medical professional.

20. A method for managing digital assets using a computer system, data storage media and a user application, the computer system having a processing device, the data storage media coupled to the computer system for storing a computer program defining an algorithm, the user application coupled to the computer system and being associated with a user device, the user application having a user interface, the processing device being in communication with the user application and including an account creation module, an event creation module and an event trigger module defined by the computer program, the method comprising the steps of:
allowing the user, using the account creation module, to create a user account using the user interface of the user application;
allowing the user, using the event creation module, to access the user account and to create an event associated with the user account, the event including an event trigger and digital asset access information associated with a digital asset; and
establishing, using the event trigger module, an indication that the event trigger has occurred and responsively providing access to the digital asset to a recipient using the digital asset access information.

21. A method, as set forth in claim 20, including the step of sending a message to the recipient when the event is created.

22. A method, as set forth in claim 21, wherein the message includes the user and the nature of the event trigger.

23. A method, as set forth in claim 20, wherein the digital asset is a digital reference to a physical item.

24. A method, as set forth in claim 23, wherein the digital reference includes a digital message associated with the physical item.

25. A method, as set forth in claim 20, including the step of proactively scanning, by the event trigger module, for the trigger event.

26. A method, as set forth in claim 20, including the step receiving, by the event trigger module, a signal from outside of the system that the event trigger has occurred.

27. A method, as set forth in claim 20, including the steps of:
allowing the user to enter a username and password and storing the username and password in a user data record associated with the user in a database,
allowing the user to enter asset data associated with the digital asset and storing the asset data in an asset record in the database.

28. A method, as set forth in claim 27, including the step of allowing the user to upload the digital asset and storing the digital asset in a digital repository.

29. A method, as set forth in claim 27, wherein the digital asset is stored in an external repository and the asset data includes a location and access details related to the digital asset in the external repository.

30. A method, as set forth in claim 20, wherein the event trigger is one or more of (a) a countdown timer, (b) a specific date and time, (c) death and (d) incapacitation.

31. A method, as set forth in claim 30, including the step of allowing, using the event creation module, the user to establish recipient data for the identified recipient for the digital asset, wherein the recipient data includes contact information.

32. A method, as set forth in claim 31, including the step of sending, by the event creation module, a communication to the recipient in response to establishment that the indication of the event trigger has occurred.

33. A method, as set forth in claim 31, wherein, if the event trigger is death, proof of death must be received by the event trigger module for the event trigger indication to be established.

34. A method, as set forth in claim 33, wherein the proof of death is manually verified.

35. A method, as set forth in claim 33, wherein the proof of death is automatically verified against an external database of death records.

36. A method, as set forth in claim 31, wherein, if the event trigger is incapacitation, then proof of incapacitation must be received by the event trigger module for the event trigger indication to be established.

37. A method, as set forth in claim 36, wherein the proof of incapacitation must be provided by a licensed medical professional.

38. A method, as set forth in claim 37, wherein the system manually or automatically confirms that the proof of incapacitation was uploaded by a medical professional.
